(19) United States
(12) Patent Application Publication
(10) Pub. No.: US 2013/0225591 A1
(43) Pub. Date: Aug. 29, 2013

(54) IMIDOTHIAZOLE KINASE INHIBITORS

(71) Applicants: Michelle R. Machacek, Brookline, MA (US); Sean P. Ahearn, Somerville, MA (US); Eric Romeo, Allston, MA (US); Tony Siu, Brookline, MA (US); Stephanie Chichetti, Brookline, MA (US); Gabriela de Almeida, Berkeley, CA (US); Alexey A. Rivkin, Emeryville, CA (US)

(72) Inventors: Michelle R. Machacek, Brookline, MA (US); Sean P. Ahearn, Somerville, MA (US); Eric Romeo, Allston, MA (US); Tony Siu, Brookline, MA (US); Stephanie Chichetti, Brookline, MA (US); Gabriela de Almeida, Berkeley, CA (US); Alexey A. Rivkin, Emeryville, CA (US)

(21) Appl. No.: 13/497,141

(22) PCT Filed: Sep. 14, 2010
(86) PCT No.: PCT/US10/48715

§ 371 Date: Apr. 30, 2013

Related U.S. Application Data
(60) Provisional application No. 61/245,414, filed on Sep. 24, 2009.

(51) Int. Cl. C07D 513/04

(52) U.S. Cl. CPC C07D 513/04
USPC 514/249; 514/252.06; 514/255.05; 514/259.1; 514/275; 514/300; 514/314; 514/338; 514/364; 514/368; 544/238; 544/281; 544/331; 544/353; 544/405; 546/121; 546/175; 546/270.1; 548/143; 548/155

(57) ABSTRACT

The present invention is directed to novel kinase inhibitors of general formula (I) and pharmaceutically acceptable salts thereof, and to the use of the kinase inhibitors of general formula (I) for treating diseases or disorders in which tau phosphorylation and cell cycle regulation is implicated, such as Alzheimer's Disease and cancer.

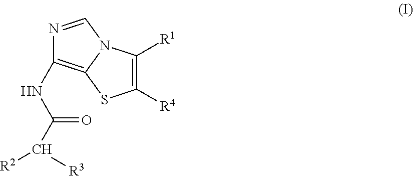

The invention is directed to tau phosphorylation and cell cycle regulation kinase inhibitors, which are useful for the treatment of Alzheimer's Disease and cancer.

### BACKGROUND OF THE INVENTION

Alzheimer's Disease is a common neurodegenerative disease affecting the elderly. Alzheimer's Disease results in progressive memory impairment, loss of language and visuospatial skills, and behavior deficits. Alzheimer's Disease is characterized by loss of mental ability severe enough to interfere with normal activities of daily living, and a marked decline in cognitive functions such as remembering, reasoning and planning. It is estimated that more than 25 million people worldwide presently suffer from Alzheimer's Disease. The number of Alzheimer's Disease patients may exceed 100 million by 2050.

Current FDA approved treatments for Alzheimer's Disease offer limited symptomatic benefits. These existing treatments target diseased neurons that release insufficient or excessive amounts of particular neurotransmitters, and seek to increase neurotransmitter levels or reduce excessive nerve cell stimulation. There are no approved pharmaceutical treatments that provide a significant delay or halt the progression of Alzheimer's Disease. Consequently, Alzheimer's Disease represents a serious unmet medical need, and many institutions are actively searching for pharmaceutical interventions for the disease.

While the cause and progression of Alzheimer's disease are not fully understood, Alzheimer's Disease is characterized by the deposition of amyloid beta (Aβ) in the brain in the form of extra-cellular plaques. This observation has led to the amyloid hypothesis, which postulates that Aβ deposits are the fundamental cause of the disease. Potential pharmaceutical interventions under the amyloid hypothesis include the prevention of Aβ formation, blocking the aggregation of amyloid into plaques, reducing amyloid solubility in the brain, and disassembling existing amyloid plaques. See Rafii et al, BMC Medicine 2 009, 7:7-11.

Typically, tau pathologies are characterized by the deposit of phosphorylated tau in the brain, abnormal conformations of tau and the presence of aggregations of tau, or “neurofibrillary tangles.” A particular characteristic of Alzheimer's Disease is the formation in the brain of neurofibrillary tangles of the tau protein. Tangles of tau are formed when hyperphosphorylated tau begins to pair with other threads of tau. The hyperphosphorylated tau forms the neurofibrillary tangles inside nerve cell bodies.

The precise role that tau plays in the pathogenesis of Alzheimer's Disease neurodegeneration is uncertain. However, tau generally promotes microtubule assembly and stabilization, and has a key role in neurogenesis, axonal maintenance and axonal transport. F. Hernandez et al, Cell Mol. Life Sci 64 (2007), 2219-2233. Tau is chiefly expressed in nerve cells.

In Alzheimer's Disease, the degree of dementia correlates more closely to the frequency of neurofibrillary tangles than to the frequency of senile plaques. Arriagada et al, Neurology 1992, 42: 631-639. In addition, tau mutations and neurofibrillary tangles are found in other dementias in which the Aβ pathology is absent, such as frontotemporal dementia, Pick's Disease and Parkinsonism linked to chromosome 17. Gong et al, J Neural Transform 2005, 112:813-838. Further, significant amounts of amyloid plaques have been found in the brains of non-demented elderly people, suggesting that amyloid pathology on its own is insufficient to cause dementia.

One potential method of inhibiting abnormal tau phosphorylation is through kinase inhibition. Cyclin dependent kinase 5 (CDK5) is a proline-directed protein kinase, which phosphorylates serine and threonine residues. CDK5 is located in the brain, and is involved in brain development. Camins et al, Drug News & Persp 2006, 8: 453-460. CDK5 has been implicated in the phosphorylation of tau. In particular, CDK5 interacts with p35, a protein which is expressed in potmitotic neurons, resulting in proteolytic products such as p25. The presence of p25 in transgenic mice is associated with hyperphosphorylation of tau. Iqbal et al, J Cell Mol Med 2008, 12:1, 38-55. Increased CDK5 activity and the accumulation of p25 is found in Alzheimer's Disease and other neurodegenerative diseases. Cruz et al, Neuron 2003, 40:471-483.

In addition to the hyperphosphorylation of tau, it is postulated that the CDK5/p25 complex induces cytoskeletal disruption, morphological degeneration and apoptosis. Thus, CDK5/p25 activation contributes to neuronal death and consequently, to neurodegenerative diseases. Camins. See also Noble et al, Neuron 2003, 40:471-483. CDK5 phosphorylation of the transient receptor potential vanilloid 1 (TRPV1) postulates a role for CDK5 in the treatment of pain. Pareek et al, PNAS 2007, 104:660-665.

Glycogen synthase kinase 3 (GSK3) is a serine/threonine protein kinase, which phosphorylates glycogen synthase. Embi, et al., Eur. J. Biochem. 1980: 107:519-527. GSK3 has also been implicated in the Alzheimer's Disease cascade, via the insulin receptor. Binding to the insulin receptor results in the activation of second messengers, including activation of the AKT protein. The AKT protein phosphorylates GSK3, leading to inactivity of GSK3.

GSK 3 exists in two isomeric forms, GSK3α and GSK3β. In addition to its role in tau phosphorylation, GSK3β regulates protection of amyloid beta in cells. Watson et al, Neurology 2003; 60(12):1899-1903. Inhibition of GSK3β is a recognized therapeutic target for Alzheimer's Diseases and other neurodegenerative diseases.

The CDK2 kinase has a role in normal cell cycling. For disorders characterized by abnormal cell cycling, such as tumors, inhibition of CDK2 may help reduce tumor growth, Inhibitors of CDK2 are therefore useful for the treatment of various types of cancer and other diseases or conditions related to abnormal cell growth See, e.g., Fischer, Cell Cycle 2004: 3(6):742-6.

### SUMMARY OF THE INVENTION

The present invention is directed to kinase inhibitors of general formula (I)

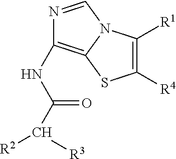

and pharmaceutically acceptable salts thereof. The compounds have been shown to inhibit tau phosphorylation kinase activity, such as CDK5 and GSK3β activity, and to inhibit cell cycle regulation activity, such as CDK2 activity.

The invention is also directed to the use of the kinase inhibitors of general formula (I) for treating diseases or disorders in which tau phosphorylation kinase inhibition and cell cycle regulation inhibition is implicated, such as Alzheimer's Disease and cancer.

### DETAILED DESCRIPTION OF THE INVENTION

In one embodiment, the present invention is directed to kinase inhibitors of general formula (I)

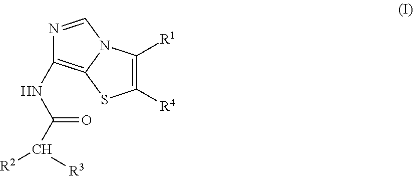

and pharmaceutically acceptable salts thereof, wherein:
R¹ is selected from the group consisting of
(1) hydrogen,
(2) halogen,
(3) cyano,
(4) —C₁₋₃ alkyl, optionally substituted with one or more fluoro, or
(5) —C₃₋₈ cycloalkyl;
   R² is selected from the group consisting of
(1) hydrogen,
(2) halogen,
(2) —C₁₋₆ alkyl, optionally substituted with hydroxyl, or
(4) hydroxyl;
   R³ is selected from the group consisting of
(1) —C₃₋₈ cycloalkyl,
(2) —C₆₋₁₀ aryl,
(3) heteroaryl, which is a cyclic or polycyclic group having 5 to 12 ring atoms, said ring atoms selected from C, C(═O), and at least one heteroatom selected from N, O or S, wherein at least one of the rings is aromatic,
(4) a heterocyclic group having 4 to 8 ring atoms, wherein one ring atom is a heteroatom selected from the group consisting of nitrogen, sulfur or oxygen, wherein said R⁵ cycloalkyl, heterocyclic, aryl or heteroaryl moiety is optionally substituted with one or more
   (a) halogen,
   (b) cyano,
   (c) —O—C₁₋₆ alkyl,
   (d) —C₁₋₆ alkyl,
   (e) OH,
   (f) —NR⁶R⁷,
   (g) heteroaryl, which is a cyclic or polycyclic group having 5 to 12 ring atoms, said ring atoms selected from C, C(═O), and at least one heteroatom selected from N, O or S, wherein at least one of the rings is aromatic,
   (h) —C₆₋₁₀ aryl,
   (i) —NH—C(═O)—R⁵,
   (j) —S(═O)₂—R⁵,
   wherein said alkyl, aryl or heteroaryl is optionally substituted with one or more
      (i) —C₁₋₆ alkyl,
      (ii) —OC₁₋₆ alkyl,
      (iii) NR⁸R⁹;
         R⁴ is selected from the group consisting of
(1) hydrogen,
(2) —C(═O)—OH,
(3) —C(═O)—NH₂;
   R⁵ is selected from the group consisting of
(1) hydrogen,
(2) —C₁₋₆ alkyl,
(3) —C₆₋₁₀ aryl,
(4) heteroaryl, which is a cyclic or polycyclic group having 5 to 12 ring atoms, said ring atoms selected from C, C(═O), and at least one heteroatom selected from N, O or S, wherein at least one of the rings is aromatic;
   R⁶, R⁷, R⁸ and R⁹ are selected from the group consisting of
(1) hydrogen,
(2) —C₁₋₆ alkyl,
(3) —C₆₋₁₀ aryl, or
(4) heteroaryl, which is a cyclic or polycyclic group having 5 to 12 ring atoms, said ring atoms selected from C, C(═O), and at least one heteroatom selected from N, O or S, wherein at least one of the rings is aromatic;
wherein said alkyl, aryl or heteroaryl R⁶, R⁷, R⁸ and R⁹ moiety is optionally substituted with one or more
   (a) halogen,
   (b) cyano,
   (c) —O—C₁₋₆ alkyl,
   (d) —C₁₋₆ alkyl,
   (e) OH,
   (f) —NR¹⁰R¹¹,
      or R⁶ and R⁷, or R⁸ and R⁹, may be linked together with the nitrogen to which they are both attached to form a non-aromatic cyclic ring having from 5 to 12 ring atoms selected from C, N, O, S, S═O or SO₂, wherein said cyclic ring is optionally substituted with one or more
(a) halogen
(b) cyano
(c) —C₃₋₈ cycloalkyl
(d) —O—C₁₋₆ alkyl,
(e) —C₁₋₆ alkyl;
   R¹⁰ and R¹¹ are selected from the group consisting of
(1) hydrogen, and
(2) —C₁₋₆ alkyl.

The invention is also directed to pharmaceutical compositions which include an effective amount of a compound of formula (I), or pharmaceutically acceptable salts thereof, and a pharmaceutically acceptable carrier.

The invention is also directed to methods of treating diseases or disorders in which tau phosphorylation kinases are implicated, such as Alzheimer's Disease, and diseases or disorders in which cell cycle regulation kinases are implicated, such as cancer, by administering a compound of formula (I), or pharmaceutically acceptable salts thereof, to a patient in need thereof.

The invention is also directed to a method for the manufacture of a medicament or a composition for the treatment of diseases or disorders in which tau phosphorylation kinases are implicated, such as Alzheimer's Disease, or for the treatment of diseases or disorders in which cell cycle regulation kinases are implicated, such as cancer, by combining a compound of the present invention with a pharmaceutical carrier or diluent.

In particular embodiments of compounds of formula (I), R¹ is selected from the group consisting of —C₁₋₃ alkyl, optionally substituted with one or more fluoro. Typically, R¹ is —CF₃. Alternatively, R¹ is hydrogen, halogen (for example, fluoro, chloro or bromo), cyano or —C₃₋₈ cycloalkyl (for example, cyclopropyl).

In particular embodiments of compounds of formula (I), R² is hydrogen. Alternatively, R² may be halogen (for example, fluoro, chloro or bromo), —C₁₋₆ alkyl (for example, methyl) optionally substituted with hydroxyl, or hydroxyl.

In particular embodiments of compounds of formula (I), R³ is aryl (for example, phenyl or napthyl), which is optionally substituted by
(a) halogen (for example, chloro),
(b) —O—C₁₋₆ alkyl (for example, methoxy),
(c) —C₁₋₆ alkyl,
(d) —NR⁶R⁷,
(e) heteroaryl, which is a cyclic or polycyclic group having 5 to 12 ring atoms, said ring atoms selected from C, C(═O), and at least one heteroatom selected from N, O or S, wherein at least one of the rings is aromatic (exemplary heteroaryl groups include pyrazolyl, pyridinyl, imidazolyl, pyridazinyl, pyrazinyl, pyrimidinyl, benzodioxol, indazolyl, quinolinyl, thienyl)
(f) —C₆₋₁₀ aryl (for example, phenyl or napthyl),
(g) —NH—C(═O)—R⁵, wherein R⁵ is aryl or heteroaryl (for example, pyridyl), or
(h) —SO₂—C₁₋₆ alkyl,
wherein said alkyl, aryl or heteroaryl is optionally substituted with one or more
   (i) —C₁₋₆ alkyl,
   (ii) —OC₁₋₆ alkyl,
   (iii) NR⁸R⁹.

In other embodiments of compounds of formula (I), R³ is heteroaryl. Suitable R³ heteroaryl groups include quinolinyl, pyridinyl, imidazolyl, imidazo[1,2-a]pyrimidinyl, imidazo[1,2-a]pyridinyl, benzodioxolyl, pyrimidinyl, indazolyl, indolyl and isoindolyl. The R³ heteroaryl groups may be substituted by
(a) halogen,
(b) —C₁₋₆ alkyl (for example, methyl),
(c) heteroaryl, which is a cyclic or polycyclic group having 5 to 12 ring atoms, said ring atoms selected from C, C(═O), and at least one heteroatom selected from N, O or S, wherein at least one of the rings is aromatic,
(d) —C₆₋₁₀ aryl (for example, phenyl or napthyl),
wherein said alkyl, aryl or heteroaryl is optionally substituted with one or more
   (i) —C₁₋₆ alkyl,
   (ii) —OC₁₋₆ alkyl,
   (iii) NR⁸R⁹.

In other embodiments of compounds of formula (I), R³ is —C₃₋₈ cycloalkyl (for example, cyclohexyl) or a heterocyclic group having 4 to 8 ring atoms, wherein one ring atom is a heteroatom selected from the group consisting of nitrogen, sulfur or oxygen (for example, tetrahydropyran), optionally substituted as described above.

In particular embodiments of compounds of formula (I), R⁴ is hydrogen. In other embodiments of compounds of formula (I), R⁴ is —C(═O)—OH or —C(═O)—NH₂.

In one subgenus, the compounds of formula (I) are compounds of formula (II):

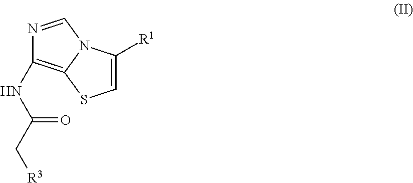

and pharmaceutically acceptable salts thereof, wherein R¹ and R³ are as described above.

In particular embodiments of the compounds of formula (II), R¹ is —C₁₋₃ alkyl, optionally substituted with one or more fluoro. Typically, R¹ is —CF₃.

Alternatively, R¹ is hydrogen, halogen (for example, fluoro, chloro or bromo), cyano or —C₃₋₈ cycloalkyl (for example, cyclopropyl).

In particular embodiments of compounds of formula (II), R³ is aryl (for example, phenyl or naphthyl), which is optionally substituted by
(a) halogen (for example, chloro),
(b) —O—C₁₋₆ alkyl (for example, methoxy),
(c) —C₁₋₆ alkyl,
(d) —NR⁶R⁷,
(e) heteroaryl, which is a cyclic or polycyclic group having 5 to 12 ring atoms, said ring atoms selected from C, C(═O), and at least one heteroatom selected from N, O or S, wherein at least one of the rings is aromatic (exemplary heteroaryl groups include pyrazolyl, pyridinyl, imidazolyl, pyridazinyl, pyrazinyl, pyrimidinyl, benzodioxol, indazolyl, quinolinyl, thienyl),
(f) —C₆₋₁₀ aryl (for example, phenyl or napthyl),
(g) —NH—C(═O)—R⁵, wherein R⁵ is aryl or heteroaryl (for example, pyridyl), or
(h) —SO₂—C₁₋₆ alkyl,
wherein said alkyl, aryl or heteroaryl is optionally substituted with one or more
   (i) —C₁₋₆ alkyl,
   (ii) —OC₁₋₆ alkyl,
   (iii) NR⁸R⁹.

In other embodiments of compounds of formula (II), R³ is heteroaryl. Suitable R³ heteroaryl groups include quinolinyl, pyridinyl, imidazolyl, imidazo[1,2-a]pyrimidinyl, imidazo[1,2-a]pyridinyl, benzodioxolyl, pyrimidinyl, indazolyl, indolyl and isoindolyl. The R³ heteroaryl groups may be substituted by
(a) halogen,
(b) —C₁₋₆ alkyl (for example, methyl),
(c) heteroaryl, which is a cyclic or polycyclic group having 5 to 12 ring atoms, said ring atoms selected from C, C(═O), and at least one heteroatom selected from N, O or S, wherein at least one of the rings is aromatic,
(d) —C₆₋₁₀ aryl (for example, phenyl or napthyl),
wherein said alkyl, aryl or heteroaryl is optionally substituted with one or more
   (i) —C₁₋₆ alkyl,
   (ii) —OC₁₋₆ alkyl,
   (iii) NR⁸R⁹.

In another subgenus, the compounds of formula (I) are compounds of formula (III):

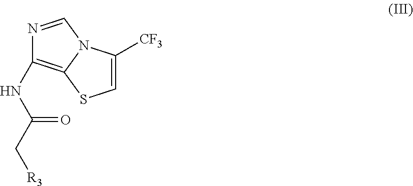

and pharmaceutically acceptable salts thereof, wherein R³ is described above.

In particular embodiments of compounds of formula (III), R³ is aryl (for example, phenyl or napthyl), which is optionally substituted by
(a) halogen (for example, chloro),
(b) —O—C₁₋₆ alkyl (for example, methoxy),
(c) —C₁₋₆ alkyl,
(d) —NR⁶R⁷,
(e) heteroaryl, which is a cyclic or polycyclic group having 5 to 12 ring atoms, said ring atoms selected from C, C(═O), and at least one heteroatom selected from N, O or S, wherein at least one of the rings is aromatic (exemplary heteroaryl groups include pyrazolyl, pyridinyl, imidazolyl, pyridazinyl, pyrazinyl, pyrimidinyl, benzodioxol, indazolyl, quinolinyl, thienyl)
(f) —C₆₋₁₀ aryl (for example, phenyl or napthyl),
(g) —NH—C(═O)—R⁵, wherein R⁵ is aryl or heteroaryl (for example, pyridyl), or
(h) —SO₂—C₁₋₆ alkyl,
wherein said alkyl, aryl or heteroaryl is optionally substituted with one or more
   (i) —C₁₋₆ alkyl,
   (ii) —OC₁₋₆ alkyl, or
   (iii) NR⁸R⁹.

In other embodiments of compounds of formula (III), R³ is heteroaryl. Suitable R³ heteroaryl groups include quinolinyl, pyridinyl, imidazolyl, imidazo[1,2-a]pyrimidinyl, imidazo[1,2-a]pyridinyl, benzodioxolyl, pyrimidinyl, indazolyl, indolyl and isoindolyl. The R³ heteroaryl groups may be substituted by
(a) halogen,
(b) —C₁₋₆ alkyl (for example, methyl),
(c) heteroaryl, which is a cyclic or polycyclic group having 5 to 12 ring atoms, said ring atoms selected from C, C(═O), and at least one heteroatom selected from N, O or S, wherein at least one of the rings is aromatic,
(d) —C₆₋₁₀ aryl (for example, phenyl or napthyl),
wherein said alkyl, aryl or heteroaryl is optionally substituted with one or more
   (i) —C₁₋₆ alkyl,
   (ii) —OC₁₋₆ alkyl, or
   (iii) —NR⁸R⁹.

The invention is also directed to pharmaceutical compositions which include an effective amount of a compound of formulas (II) or (III), or pharmaceutically acceptable salts thereof, and a pharmaceutically acceptable carrier.

The invention is also directed to methods of treating diseases or disorders in which tau phosphorylation kinases are implicated, such as Alzheimer's Disease, and diseases or disorders in which cell cycle regulation kinases are implicated, such as cancer, by administering a compound of formulas (II) or (III), or pharmaceutically acceptable salts thereof, to a patient in need thereof.

The invention is also directed to a method for the manufacture of a medicament or composition for the treatment of diseases or disorders in which tau phosphorylation kinases are implicated, such as Alzheimer's Disease, or diseases or disorders in which cell cycle regulation kinases are implicated, such as cancer, by combining a compound of one of formulas (II) or (III) with a pharmaceutical carrier or diluent.

As used herein, the term “alkyl,” by itself or as part of another substituent, means a saturated straight or branched chain hydrocarbon radical having the number of carbon atoms designated (e.g., C₁₋₁₀ alkyl means an alkyl group having from one to ten carbon atoms). Preferred alkyl groups for use in the invention are C₁₋₆ alkyl groups, having from one to six carbon atoms. Exemplary alkyl groups include methyl, ethyl, n-propyl, isopropyl, n-butyl, isobutyl, tert-butyl, pentyl, hexyl, and the like. C₀ alkyl means a bond.

As used herein, the term “cycloalkyl,” by itself or as part of another substituent, means a saturated cyclic hydrocarbon radical having the number of carbon atoms designated (e.g., C₃₋₁₂ cycloalkyl means a cycloalkyl group having from three to twelve carbon atoms). The term cycloalkyl as used herein includes mono-, bi- and tricyclic saturated carbocycles, as well as bridged and fused ring carbocycles, such as spiro fused ring systems.

Preferred cycloalkyl groups for use in the invention are monocyclic C₃₋₈ cycloalkyl groups, having from three to eight carbon atoms. Exemplary monocyclic cycloalkyl groups include cyclopropyl, cyclobutyl, cyclopentyl, cyclohexyl and the like. Exemplary bridged cycloalkyl groups include adamantly and norbornyl. Exemplary fused cycloalkyl groups include decahydronaphthalene.

When a non-aromatic heterocyclic group as defined herein is substituted, the substituent may be bonded to a ring carbon atom of the heterocyclic group, or on a ring heteroatom (i.e., a nitrogen, oxygen or sulfur), which has a valence which permits substitution. Preferably, the substituent is bonded to a ring carbon atom. Similarly, when a non-aromatic heterocyclic group is defined as a substituent herein, the point of attachment may be at a ring carbon atom of the heterocyclic group or on a ring heteroatom (i.e., a nitrogen, oxygen or sulfur), which has a valence which permits substitution. Preferably, the attachment is at a ring carbon atom.

As used herein, the term “aryl,” by itself or as part of another substituent, means an aromatic cyclic hydrocarbon radical having the number of carbon atoms designated (e.g., C₆₋₁₀ aryl means an aryl group having from six to ten carbons atoms). The term “aryl” includes multiple ring systems as well as single ring systems. Preferred aryl groups for use in the invention include phenyl and naphthyl.

The term “aryl” also includes fused cyclic hydrocarbon rings which are partially aromatic (i.e., one of the fused rings is aromatic and the other is non-aromatic). An exemplary aryl group which is partially aromatic is indanyl.

The term “halo” or “halogen” includes fluoro, chloro, bromo and iodo.

As used herein, the term “heteroaryl,” by itself or as part of another substituent, means an aromatic cyclic group having at least one ring heteroatom (O, N or S). The term “heteroaryl” includes multiple ring systems as well as single ring systems. Exemplary heteroaryl groups for use in the invention include furyl, pyranyl, benzofuranyl, isobenzofuranyl, chromenyl, thienyl, benzothiophenyl, pyrrolyl, pyrazolyl, imidazolyl, pyridyl, pyrazinyl, pyrimidinyl, pyridazinyl, indolyl, benzimidazolyl, quinolinyl, isoquinolinyl, tetrazolyl, indazolyl, napthyridinyl, triazolyl, oxazolyl, oxadiazolyl, thiazolyl, thiadiazolyl, isoxazolyl, tetrahydroquinolinyl, tetrahydroisoquinolinyl and dihydroindolyl.

The term “heteroaryl” also includes fused aromatic cyclic groups which are partially aromatic one of the fused rings is aromatic and the other is non-aromatic). Exemplary heteroaryl groups which are partially aromatic include tetrahydroquinolyl, dihydrobenzofuran and dihydroindolyl.

When a heteroaryl group as defined herein is substituted, the substituent may be bonded to a ring carbon atom of the heteroaryl group, or on a ring heteroatom (i.e., a nitrogen, oxygen or sulfur), which has a valence which permits substitution. Preferably, the substituent is bonded to a ring carbon atom. Similarly, when a heteroaryl group is defined as a substituent herein, the point of attachment may be at a ring carbon atom of the heteroaryl group, or on a ring heteroatom a nitrogen, oxygen or sulfur), which has a valence which permits attachment. Preferably, the attachment is at a ring carbon atom.

Some of the compounds of the instant invention have at least one asymmetric center. Additional asymmetric centers may be present depending upon the nature of the various substituents on the molecule. Compounds with asymmetric centers give rise to enantiomers (optical isomers), diastereomers (configurational isomers) or both, and it is intended that all of the possible enantiomers and diastereomers in mixtures and as pure or partially purified compounds are included within the scope of this invention. The present invention is meant to encompass all such isomeric forms of these compounds.

Compounds described herein may contain one or more double bonds, and may thus give rise to cis/trans isomers as well as other conformational isomers. The present invention includes all such possible isomers as well as mixtures of such isomers.

Formulas (I) to (III) are shown above without a definite stereochemistry at certain positions. The present invention includes all stereoisomers of formulas (I) to (III) and pharmaceutically acceptable salts thereof.

In the compounds of formulas (I) to (III), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic formulas (I) to (III). For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic formulas (I) to (III) can be prepared without undue experimentation by conventional techniques well known to those skilled in the art or by processes analogous to those described in the schemes and examples herein using appropriate isotopically-enriched reagents and/or intermediates.

The term “substantially pure” means that the isolated material is at least 90% pure, and preferably 95% pure, and even more preferably 99% pure as assayed by analytical techniques known in the art.

The term “pharmaceutically acceptable salts” refers to salts prepared from pharmaceutically acceptable non-toxic bases or acids including inorganic or organic bases and inorganic or organic acids. The compounds of the invention may be mono, di or tris salts, depending on the number of acid functionalities present in the free base form of the compound. Free bases and salts derived from inorganic bases include aluminum, ammonium, calcium, copper, ferric, ferrous, lithium, magnesium, manganic salts, manganous, potassium, sodium, zinc, and the like. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, trifluoroacetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

The subject or patient to whom the compounds of the present invention is administered is generally a human being, male or female, in whom inhibition of tau phosphorylation or cell cycle regulation kinase activity is desired, but may also encompass other mammals, such as dogs, cats, mice, rats, cattle, horses, sheep, rabbits, monkeys, chimpanzees or other apes or primates, for which inhibition of kinase activity or treatment of the above noted disorders is desired.

The compounds of the invention are useful for treating diseases or disorders in which tau phosphorylation and cell cycle regulation kinases are implicated, such as Alzheimer's Disease and other neurodegenerative diseases. Neurodegenerative diseases in which CDK5 is implicated include mild cognitive impairment; age-related cognitive decline; corticobasal degeneration; dementia pugilistica; Down's Syndrome; frontotemporal dementia; Parkinson's Disease and Parkinsonism linked to chromosome 17; Parkinsonian-ALS demential complex; cerebral ischemia and other strokes; spinal cord injury; traumatic brain injury; viral induced dementia, such as HIV and AIDS induced dementia; excitotoxicity; epilepsy; amyotrophic lateral sclerosis; Niemann-Pick type C disease; neurodegeneration due to myocardial infarction and oxidative stresses; Huntington's Disease and dementia due to Huntington's disease; myotonic dystrophy; priori disease with tangles; progressive supranuclear palsy; lower lateral sclerosis; sucabcute sclerosing panencephalistis; multiple sclerosis; neurodegeneration associated with bacterial infection, migraine, hypoglycemia, urinary incontinence, brain ischemia, and emesis. The compounds of the invention are also useful in the treatment of pain.

In addition, the compounds of the invention may be useful for treating schizophrenia; schizophreniform disorder; schizoaffective disorder, for example of the delusional type or the depressive type; delusional disorder; substance-induced psychotic disorder, for example psychosis induced by alcohol, amphetamine, cannabis, cocaine, hallucinogens, inhalants, opioids, or phencyclidine; personality disorder of the paranoid type; personality disorder of the schizoid type; drug addiction, including narcotic (e.g. heroin, opium, and morphine), cocaine and alcohol addiction; drug withdrawal, including narcotic, cocaine and alcohol withdrawal; obsessive compulsive disorder; Tourette's syndrome; depression; a major depressive episode, a manic or mixed mood episode, a hypomanic mood episode, a depressive episode with atypical features or with melancholic features or catatonic features, a mood episode with postpartum onset; post-stroke depression, major depressive disorder, dysthymic disorder, minor depressive disorder, premenstrual dysphoric disorder, post-psychotic depressive disorder of schizophrenia, a major depressive disorder superimposed on a psychotic disorder such as delusional disorder or schizophrenia, a bipolar disorder, for example bipolar I disorder, bipolar II disorder, cyclothymic disorder; anxiety; attention deficit and hyperactivity disorder; and attention deficit disorder.

Other disorders and conditions for which the compounds of the invention may be useful include male fertility and sperm motility; diabetes mellitus; impaired glucose tolerance; metabolic syndrome or syndrome X; polycystic ovary syndrome; adipogenesis and obesity; myogenesis and frailty, for example age-related decline in physical performance; acute sarcopenia, for example muscle atrophy and/or cachexia associated with burns, bed rest, limb immobilization, or major thoracic, abdominal, and/or orthopedic surgery; sepsis; hair loss, hair thinning, and balding; and immunodeficiency

For example, the compounds may be useful for the prevention of dementia of the Alzheimer's type, as well as for the treatment of early stage, intermediate stage or late stage dementia of the Alzheimer's type. In general, Alzheimer's Disease symptoms include confusion, irritability and aggression, mood swings, language breakdown, long-term memory loss, and the general withdrawal of the sufferer as their senses decline. The language problems associated with Alzheimer's Disease include a shrinking vocabulary and decreased word fluency. Alzheimer's Disease also includes impairment of fine motor tasks, such as writing, drawing, dressing and other coordinated movements. Alzheimer's Disease symptoms include apraxia (difficulties in movement planning).

Early stage Alzheimer's Disease is characterized by confusion, memory loss and changes in other cognitive abilities. Symptoms may include getting lost, trouble handling money and paying bills, repeating questions, taking longer to complete normal daily tasks, poor judgment, and mood and personality changes.

Intermediate stage Alzheimer's Disease is manifested by problems with reasoning, sensory processing, and conscious thought. Intermediate stage symptoms include continuing memory loss and confusion. Intermediate stage patients typically begin to have problems recognizing family and friends. Symptoms include the inability to learn new things, carry out tasks that involve multiple steps (such as getting dressed), or coping with new situations. Intermediate stage patients may have hallucinations, delusions, and paranoia, and may behave impulsively.

Patients suffering from severe Alzheimer's Disease are typically unable to communicate and are completely dependent on others for their care.

The compounds of the invention are used to treat or prevent cellular proliferation diseases. Cellular proliferation disease states include, but are not limited to, cancer (further discussed below), autoimmune disease, arthritis, graft rejection, inflammatory bowel disease, proliferation induced after medical procedures, including, but not limited to, surgery, angioplasty, and the like. It is appreciated that in some cases the cells may not be in a hyper- or hypoproliferation state (abnormal state) and still require treatment. Thus, in one embodiment, the invention herein includes application to cells or individuals which are afflicted or may eventually become afflicted with any one of these disorders or states.

The compounds, compositions and methods provided herein are particularly useful for the treatment and prevention of cancer, such as angiogenesis and tumorigenesis, and including the treatment of solid tumors such as skin, breast, brain, cervical carcinomas, testicular carcinomas, and the like. Particular cancers that may be treated by the compounds, compositions and methods of the invention include, but are not limited to cardiac sarcomas: angiosarcoma, fibrosarcoma, rhabdomyosarcoma, liposarcoma, myxoma, rhabdomyoma, fibroma, lipoma and teratoma; lung sarcomas: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal sarcomas: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); genitourinary tract sarcomas: kidney (adenocarcinoma, Wilm's tumor or nephroblastoma, lymphoma, leukemia,), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver sarcomas: hepatoma (hepatocellular carcinoma), cholangiocarcinoma, hepatoblastoma, angiosarcoma, hepatocellular adenoma, hemangioma; bone sarcomas: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system sarcomas: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord (neurofibroma, meningioma, glioma, sarcoma); gynecological sarcomas: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); hematologic sarcomas: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); skin sarcomas: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. The term “cancerous cell” as provided herein, includes a cell afflicted by any one of the above-identified conditions.

In another embodiment, the compounds of the instant invention are useful for treating or preventing cancer selected from head and neck squamous cell carcinomas, histiocytic lymphoma, lung adenocarcinoma, small cell lung cancer, non-small cell lung cancer, pancreatic cancer, papillary renal cell carcinoma, liver cancer, gastric cancer, colon cancer, multiple myeloma, glioblastomas and breast carcinoma. In another embodiment, the compounds of the instant invention are useful for the prevention or modulation of the metastases of cancer cells and cancer.

The compounds of the present invention may be used in combination with one or more other drugs in the treatment of diseases or conditions for which the compounds of the present invention have utility, where the combination of the drugs together are safer or more effective than either drug alone. Additionally, the compounds of the present invention may be used in combination with one or more other drugs that treat, prevent, control, ameliorate, or reduce the risk of side effects or toxicity of the compounds of the present invention. Such other drugs may be administered, by a route and in an amount commonly used therefor, contemporaneously or sequentially with the compounds of the present invention. Accordingly, the pharmaceutical compositions of the present invention include those that contain one or more other active ingredients, in addition to the compounds of the present invention. The combinations may be administered as part of a unit dosage form combination product, or as a kit or treatment protocol wherein one or more additional drugs are administered in separate dosage forms as part of a treatment regimen.

Examples of combinations of the compounds of the present invention include combinations with anti-Alzheimer's Disease agents, for example other CDK5 inhibitors; beta-secretase inhibitors; alpha 7 nicotinic agonists; ADAM 10 ligands or activators; gamma-secretase inhibitors; gamma secretase modulators; tau phosphorylation inhibitors; glycine transport inhibitors; LXR β agonists; ApoE4 conformational modulators; NR2B antagonists; androgen receptor modulators; blockers of Aβ oligomer formation; 5-HT4 agonists; 5-HT6 antagonists; 5-HT1a antagonists, such as lecozotan; NK1/NK3 receptor antagonists; COX-2 inhibitors; HMG-CoA reductase inhibitors; NSAIDs including ibuprofen; vitamin E; anti-amyloid antibodies (including anti-amyloid humanized monoclonal antibodies); anti-inflammatory compounds such as (R)-flurbiprofen, nitroflurbiprofen; PPAR gamma agonists, such as pioglitazone and rosiglitazone; CB-1 receptor antagonists or CB-1 receptor inverse agonists; antibiotics such as doxycycline and rifampin; N-methyl-D-aspartate (NMDA) receptor antagonists, such as memantine, neramexane; cholinesterase inhibitors such as galantamine, rivastigmine, donepezil, tacrine, phenserine and ladostigil; growth hormone secretagogues such as ibutamoren, ibutamoren mesylate, and capromorelin; histamine H₃ receptor antagonists; AMPA agonists or AMPA modulators; PDE 4 inhibitors; PDE 10A inhibitors; GABA_{A} inverse agonists; GSK3β inhibitors; neuronal nicotinic agonists; selective M1 agonists; HDAC inhibitors; and microtubule affinity regulating kinase (MARK) ligands; or other drugs that affect receptors or enzymes that either increase the efficacy, safety, convenience, or reduce unwanted side effects or toxicity of the compounds of the present invention.

The instant compounds are also useful in combination with known anti-cancer agents. For example, the compounds are useful in combination with known anti-cancer agents. Examples of such agents can be found in Cancer Principles and Practice of Oncology by V. T. Devita and S. Hellman (editors), 6th edition (2001). Suitable anti-cancer agents include, but are not limited to, estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, and apoptosis inducing agents and agents that interfere with cell cycle checkpoints.

The instant compounds are also useful when co-administered with radiation therapy.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate and 4,4′-dihydroxybenzophenone-2,4-dinitrophenyl-hydrazone.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, trans-N-(4′-hydroxyphenyl)retinamide and N-4-carboxyphenyl retinamide.

“Cytotoxic/cytostatic agents” refer to compounds which cause cell death or inhibit cell proliferation primarily by interfering directly with the cell's functioning or inhibit or interfere with cell mytosis, including alkylating agents, tumor necrosis factors, intercalators, hypoxia activatable compounds, microtubule inhibitors/microtubule-stabilizing agents, inhibitors of mitotic kinesins, inhibitors of histone deacetylase, inhibitors of kinases involved in mitotic progression, antimetabolites, biological response modifiers, hormonal/anti-hormonal therapeutic agents, haematopoietic growth factors, monoclonal antibody targeted therapeutic agents, topoisomerase inhibitors, proteasome inhibitors and ubiquitin ligase inhibitors. Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPXIOO, (trans, trans, trans)-bis-mu-(hexane-1,6-diamine)-mu-[diamine-platinum(π)]bis[diamine(chloro)platinu πi (II)]tetrachloride, diarizidinylspermine, arsenic trioxide, 1-(1 l-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755 and 4-demethoxy-3-deamino-3-aziridinyl-4-methylsulphonyl-daunorubicin.

An example of a hypoxia activatable compound is tirapazamine.

Examples of proteasome inhibitors include but are not limited to lactacystin and bortezomib.

Examples of microtubule inhibitors/microtubule-stabilising agents include paclitaxel, vindesine sulfate, 3′,4-didehydro-4′-deoxy-8′-norvincaleukoblastine, docetaxol, rhizoxin, dolastatin, mivobulin isethionate, auristatin, cemadotin, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide and epothilones.

Examples of topoisomerase inhibitors are topotecan, hycaptamine, irinotecan, rubitecan, 6-ethoxypropionyl-3′,4′-0-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, etoposide phosphate, teniposide, sobuzoxane, T-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydro0xy-3,5-dimethoxyphenyl]-55a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N—[I-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one and dimesna.

Examples of inhibitors of mitotic kinesins include, but are not limited to inhibitors of KSP, inhibitors of MKLP1, inhibitors of CENP-E, inhibitors of MCAK, inhibitors of Kif14, inhibitors of Mphosph1 and inhibitors of Rab6-KIFL.

Examples of “histone deacetylase inhibitors” include, but are not limited to SAHA, TSA, oxamflatin, PXD1O1, MG98, valproic acid and scriptaid. Further reference to other histone deacetylase inhibitors are described in Miller, T. A. et al. J. Med. Chem. 46(24):5097-51 16 (2003).

“Inhibitors of kinases involved in mitotic progression” include, but are not limited to, inhibitors of aurora kinase, inhibitors of Polo-like kinases (PLK) (in particular inhibitors of PLK-I), inhibitors of bub-1 and inhibitors of bub-R1.

“Antiproliferative agents” includes antisense RNA and DNA oligonucleotides, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

“HMG-CoA reductase inhibitors” refers to inhibitors of 3-hydroxy-3-methylglutaryl-CoA reductase. Examples of HMG-CoA reductase inhibitors that may be used include, but are not limited to lovastatin, simvastatin, pravastatin, fluvastatin and atorvastatin. The structural formulas of these and additional HMG-CoA reductase inhibitors that may be used in the instant methods are described at page 87 of M. Yalpani, “Cholesterol Lowering Drugs”, Chemistry & Industry, pp. 85-89 (1996). The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-H, also called Rab GGPTase).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors FIt-I (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal antiinflammatories (NSAIDs) like aspirin and ibuprofen as well as selective cyclooxy-genase-2 inhibitors like celecoxib and rofecoxib, steroidal antiinflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagi πol, thalidomide, angiostatin, troponin-1, angiotensin II antagonists and antibodies to VEGF.

Other therapeutic agents that modulate or inhibit angiogenesis and may also be used in combination with the compounds of the invention include agents that modulate or inhibit the coagulation and fibrinolysis systems (see Clin. Chem. La. Med. 38:679-692 (2000)). Examples of such agents that modulate or inhibit the coagulation and fibrinolysis pathways include, but are not limited to, heparin, low molecular weight heparins and carboxypeptidase U inhibitors (also known as inhibitors of active thrombin activatable fibrinolysis inhibitor).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” refer to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs as described by Bume-Jensen et al, Nature 2001; 4 11-0.355-365.

“Inhibitors of cell proliferation and survival signaling pathway” refer to pharmaceutical agents that inhibit cell surface receptors and signal transduction cascades downstream of those surface receptors. Such agents include inhibitors of EGFR (for example gefitinib and erlotinib), inhibitors of ERB-2 (for example trastuzumab), inhibitors of IGFR, inhibitors of cytokine receptors, inhibitors of MET, inhibitors of PDK, serine/threonine kinases (including but not limited to inhibitors of Akt, inhibitors of Raf kinase, inhibitors of MEK and inhibitors of mTOR. Such agents include small molecule inhibitor compounds and antibody antagonists.

“Apoptosis inducing agents” include activators of TNF receptor family members (including the TRAIL receptors).

The invention also encompasses combinations with NSAID's which are selective COX-2 inhibitors. For purposes of this specification, NSAID's which are selective inhibitors of COX-2 are defined as those which possess a specificity for inhibiting COX-2 over COX-1 of at least 100 fold as measured by the ratio of IC50 for COX-2 over IC50 for COX-1 evaluated by cell or microsomal assays. Inhibitors of COX-2 that are particularly useful in the instant method of treatment are 3-phenyl-4-(4-(memylsulfonyl)phenyl)-2-(5/0-furanone; 5-chloro-3-(4-methylsulfonyl)-phenyl-2-(2-methyl-5-pyridinyl)pyridine; or a pharmaceutically acceptable salt thereof.

Compounds that have been described as specific inhibitors of COX-2 and are therefore useful in the present invention include, but are not limited to: parecoxib, CELEBREX and BEXTRA or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)-phenyl]methyl]-IH-1,2,3-triazole-4-carboxamide, CM 101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate) and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αv β3 integrin, to compounds which selectively antagonize, inhibit or counteract binding of a physiological ligand to the αvβ5 integrin, to compounds which antagonize, inhibit or counteract binding of a physiological ligand to both the αyβ3 integrin and the αv β5 integrin, and to compounds which antagonize, inhibit or counteract the activity of the particular integrin(s) expressed on capillary endothelial cells. The term also refers to antagonists of the αv β6, αγβ8 cti βi, 2β1<*5β1 α6β1 and αβ4 integrins. The term also refers to antagonists of any combination of αβ3 αv β5, cx v β6, ctv β8 αi βi, α2β1, αs β1, αββ1 and 6β4 integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin>4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, 2,3,9,10,11,12-hexahydro-10-(hydroxymethyl)-10-hydroxy-9-methyl-9,12-epoxy-1H-diindolo[1,2,3-fg:3′,2′,r-kl]pyrrolo[3,4-i][1,6]benzodiazocin-1-one, SH268, genistein, imatinib, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine and EMD 12 1974.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients.

In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound, which is a compound of formulas (I) to (III), is included in an amount sufficient to produce the desired effect upon the process or condition of diseases. Accordingly, the pharmaceutical compositions of the present invention encompass any composition made by admixing a compound of the present invention and a pharmaceutically acceptable carrier.

The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds represented by formulas (I) to (III), or pharmaceutically acceptable salts thereof, may also be administered by controlled release means and/or delivery devices.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient.

Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Other pharmaceutical compositions include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the invention may also be in the form of oil-in-water emulsions, which may also contain excipients such as sweetening and flavoring agents.

The pharmaceutical compositions may be in the form of a sterile injectable aqueous or oleaginous suspension, or in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can also be in a form suitable for rectal administration wherein the carrier is a solid. It is preferable that the mixture forms unit dose suppositories. Suitable carriers include cocoa butter and other materials commonly used in the art.

By “pharmaceutically acceptable” it is meant the carrier, diluent or excipient must be compatible with the other ingredients of the formulation and not deleterious to the recipient thereof.

The terms “administration of” or “administering a” compound should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The terms “effective amount” or “therapeutically effective amount” means the amount of the subject compound that will elicit the biological or medical response of a tissue, system, animal or human that is being sought by the researcher, veterinarian, medical doctor or other clinician.

As used herein, the term “treatment” or “treating” means any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

The compositions containing compounds of the present invention may conveniently be presented in unit dosage form and may be prepared by any of the methods well known in the art of pharmacy. The term “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets or capsules for oral administration, single dose vials for injection, or suppositories for rectal administration. This list of unit dosage forms is not intended to be limiting in any way, but merely to represent typical examples of unit dosage forms.

The compositions containing compounds of the present invention may conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient.

When treating or ameliorating Alzheimer's disease or cancer, or other diseases for which compounds of the present invention are indicated, generally satisfactory results are obtained when the compounds of the present invention are administered at a daily dosage of from about 0.1 mg to about 100 mg per kg of animal body weight, preferably given as a single daily dose or in divided doses two to six times a day, or in sustained release form. The total daily dosage is from about 1.0 mg to about 2000 mg, preferably from about 0.1 mg to about 20 mg per kg of body weight. In the case of a 70 kg adult human, the total daily dose will generally be from about 7 mg to about 1,400 mg. This dosage regimen may be adjusted to provide the optimal therapeutic response. The compounds may be administered on a regimen of 1 to 4 times per day, preferably once or twice per day.

The amount of active ingredient that may be combined with the carrier materials to produce a single dosage form will vary depending upon the host treated and the particular mode of administration. For example, a formulation intended for the oral administration to humans may conveniently contain from about 0.005 mg to about 2.5 g of active agent, compounded with an appropriate and convenient amount of carrier material. Unit dosage forms will generally contain between from about 0.005 mg to about 1000 mg of the active ingredient, typically 0.005, 0.01 mg, 0.05 mg, 0.25 mg, 1 mg, 5 mg, 25 mg, 50 mg, 100 mg, 200 mg, 300 mg, 400 mg, 500 mg, 600 mg, 800 mg or 1000 mg, administered once, twice or three times a day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The independent syntheses of the enantiomerically or diastereomerically enriched compounds, or their chromatographic separations, may be achieved as known in the art by appropriate modification of the methodology disclosed herein. Their absolute stereochemistry may be determined by the x-ray crystallography of crystalline products or crystalline intermediates that are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods using chiral stationary phases, which methods are well known in the art.

Alternatively, any enantiomer of a compound may be obtained by stereoselective synthesis using optically pure starting materials or reagents of known configuration by methods well known in the art.

In some cases the final product may be further modified, for example, by manipulation of substituents. These manipulations may include, but are not limited to, reduction, oxidation, alkylation, acylation, and hydrolysis reactions which are commonly known to those skilled in the art.

In some cases the order of carrying out the foregoing reaction schemes may be varied to facilitate the reaction or to avoid unwanted reaction products. Additionally, various protecting group strategies may be employed to facilitate the reaction or to avoid unwanted reaction products.

The compounds claimed in this invention can be prepared according to the following general procedure methods (Schemes 1-3).

### Methods of Synthesis

### Method 1

General procedures to prepare compounds of the instant invention are described in Scheme 1. 4,5-Dinitro-1H-imidazole (I) can be reacted with sodium sulfide to afford thiol derivative II. The thiol derivative can be alkylated with an alpha bromoketone (III) in the presence of a tertiary phosphine to afford thioether IV. The thioether can be cyclized in the presence of phosphorous oxychloride to afford imidothiazole V, which can be reduced in the presence of hydrogen and palladium on carbon, followed by acidification with HCl or other Bronsted acids to afford amino compound VI as a salt. The amino compound VI can be elaborated to the final product VII through an amide coupling using EDC or another appropriate amide coupling reagent with an appropriately substituted carboxylic acid.

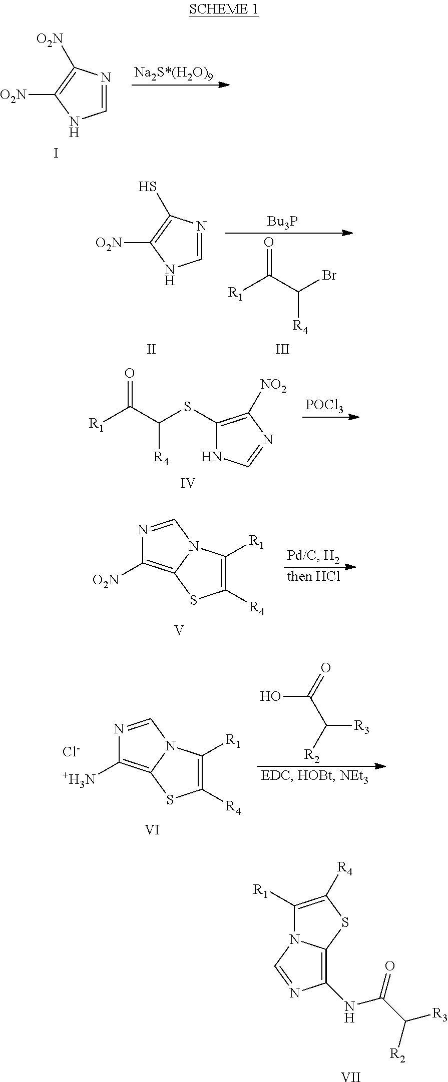

### Method 2

General procedures to prepare compounds of the instant invention are also described in Scheme 2. Aminomethyl thiazole VIII can be reacted with various formylating agents known to those skilled in the art such as ethyl formate at reflux to afford formamide IX. This formamide can be cyclized in the presence of phosphorous oxychloride at reflux or several other dehydrating reagents to afford imidazothiazole X. Nitration in the presence of nitronium tetrafluoroborate at low temperature or using other nitrating reagent affords nitro derivative V. Reduction of the nitro group in the presence of hydrogen using platinum on carbon or other reducing conditions affords amino derivative VI. The amino compound VI can be elaborated to the final product VII by treatment with an appropriately substituted acid chloride.

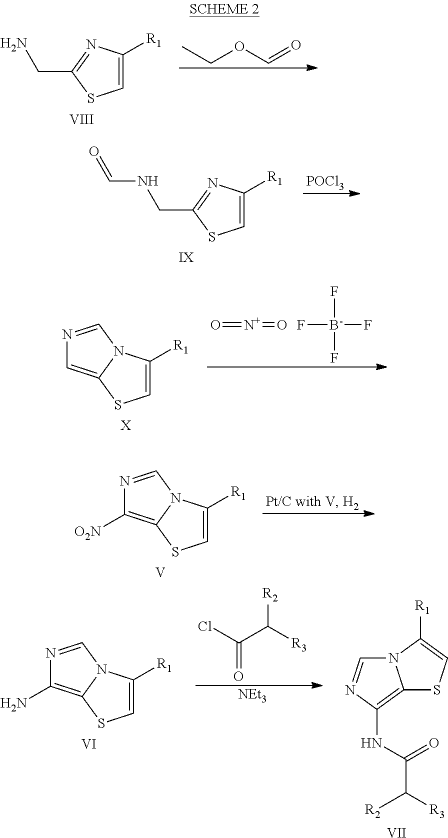

### Method 3

General procedures to prepare compounds of the instant invention are also described in Scheme 3. Imidazothiazole X can be halogenated with various halogenating agents known to those skilled in the art such as N-iodosuccinimide to afford iodo derivative XI. This iodo-compound can be cross-coupled with appropriately substituted amides in the presence of copper (I) iodide and di-amine ligands at high temperature to afford the final product VII.

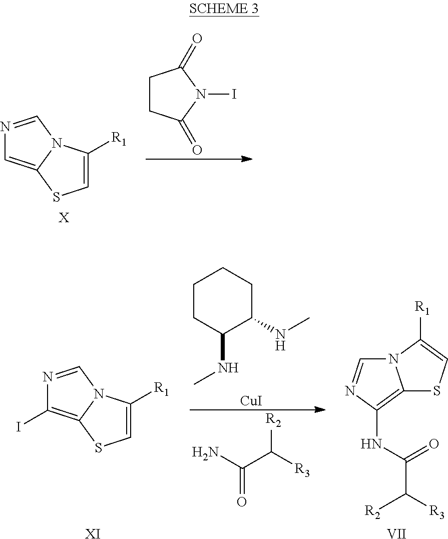

The following Examples are provided so that the invention might be more fully understood. These Examples are illustrative only and should not be construed as limiting the invention in any way.

### Example 1

### 2-[4-(2-Oxo-1,3-oxazolidin-3-yl)phenyl]-N-[3-(trifluoromethyl)imidazo[5,1-b][1,3]thiazol-7-yl]acetamide (6)

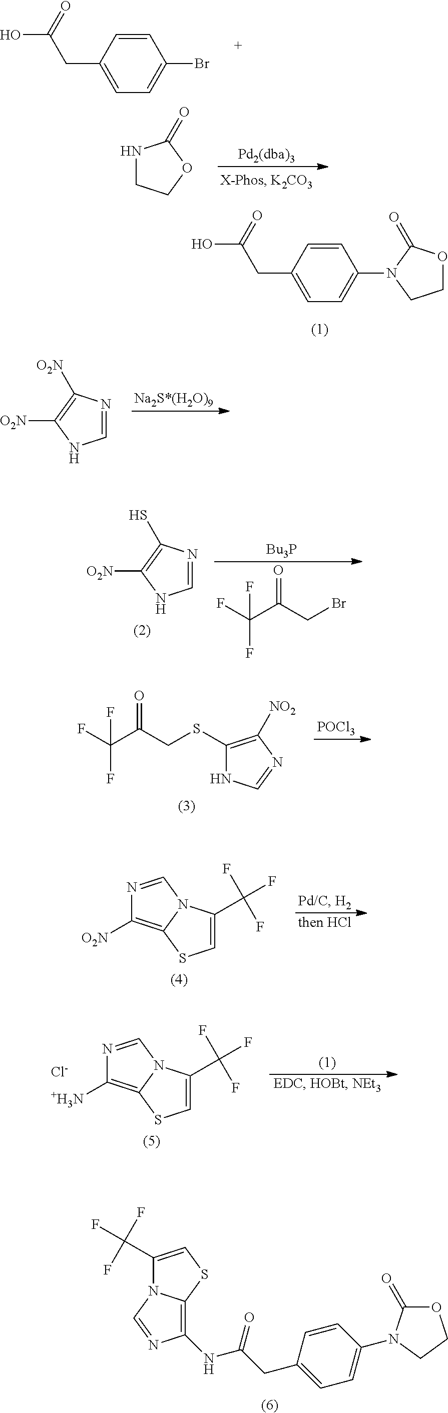

### Step 1: [4-(2-Oxo-1,3-oxazolidin-3-yl)phenyl]acetic Acid (1)

4-Bromophenylacetic acid (1 g, 4.7 mmol), 2-oxazolidinone (0.41 g, 4.7 mmol), X-PHOS (0.22 g, 0.47 mmol), potassium carbonate (1.93 g, 14 mmol), and Pd₂(dba)₃ (0.21 g, 0.23 mmol) were placed in a vial that was subsequently evacuated and backfilled with argon (3×). Fully degassed tert-amyl alcohol (23 ml) was then added and the reaction was heated to 90° C. and stirred overnight under argon. The reaction was then allowed to cool to room temperature, filtered, and the filtrate was diluted with dichloromethane and water. The organic layer was separated, and the aqueous layer was filtered once more. The aqueous filtrate was then acidified to pH 3 with aqueous hydrochloric acid and extracted with dichloromethane (3×). The organic layer was then washed with brine, dried over magnesium sulfate, filtered, and concentrated under reduced pressure. The resulting residue was dissolved in a hot dichloromethane and ethyl acetate and the title product was crashed out with hexanes. LRMS (APCI) calc'd for C₁₁H₁₁NO₄ [M+H]⁺: 222.1, Found: 221.9. ¹H NMR (600 MHz, d6-DMSO): δ 12.28 (bs, 1H), 7.48 (d, 2H), 7.25 (d, 2H), 4.41 (t, 2H), 4.03 (t, 2H), 3.52 (s, 2H).

### Step 2: 5-Nitro-1H-imidazole-4-thiol (2)

4,5-Dinitro-1H-imidazole (15 g, 95 mmol) was dissolved in water (470 ml), and sodium sulfate nonahydrate (45.6 g, 190 mmol) was added in portions. The reaction mixture was stirred over 70 minutes at room temperature and then acidified with 6M HCl to pH 3. The formed precipitate was collected by filtration, washed with cold water, and then washed with cold acetone to afford the title compound. ¹H NMR (600 MHz, d6-DMSO): δ 13.63 (bs, 1H), 8.01 (s, 1H).

### Step 3: 1,1,1-Trifluoro-3-[(4-nitro-1H-imidazole-5-yl)thio]acetone (3)

5-Nitro-1H-imidazole-4-thiol (8.7 g, 60 mmol) was placed in a vial that was then evacuated and backfilled with argon (3×). DMF (300 ml) and tributylphosphine (7.4 ml, 30 mmol) were then added and the reaction stirred at room temperature for one hour. 3-Bromo-1,1,1-trifluoroacetone (12.6 g, 66 mmol) was then added and the reaction stirred at room temperature overnight. The mixture was then concentrated under reduced pressure and azeotroped with toluene two times. The resulting thick slurry was taken up in dichloromethane with a little bit of ethyl acetate and purified via silica gel chromatography (0-50% ethyl acetate in dichloromethane). Fractions containing the title compound were concentrated and then azeotroped two times with toluene. The resulting thick slurry was taken up in ethyl acetate until fully dissolved, and then triturated using hexanes to afford the title compound as a light blue powder. ¹H NMR (600 MHz, d6-DMSO): δ 9.68 (s, 1H), 8.10 (s, 1H), 4.55 (d, 1H), 3.71 (d, 1H).

### Step 4: 7-Nitro-3-(trifluoromethyl)imidazo[5,1-b][1,3]thiazole (4)

1,1,1-Trifluoro-3-[(4-nitro-1H-imidazole-5-yl)thio]acetone (5.1 g, 20 mmol) was taken up in phosphorous oxychloride (37 ml, 400 mmol) and refluxed at 100° C. overnight. The reaction was then poured into ice water and a small amount of acetonitrile. Aqueous sodium bicarbonate was added until pH 7. The precipitate was collected by filtration, slurried in dichloromethane, ethyl acetate, and methanol. The solids were collected by filtration to yield the title compound as a light brown solid. The aqueous sodium bicarbonate filtrate was concentrated under reduced pressure, taken up in ethyl acetate, dichloromethane, and methanol and heated. The suspension was filtered to remove the insoluble salts. The filtrate was concentrated and then taken up in ethyl acetate, dichloromethane, and methanol and the insoluble material was filtered out. The filtrate was concentrated down to about ½ volume and purified via silica gel chromatography (0-20% ethyl acetate in dichloromethane) to afford a second portion of the title compound. LRMS (APCI) calc'd for C₆H₂F₃N₃O₂S [M+H]⁺: 238.0, Found: 237.8. ¹H NMR (600 MHz, d6-DMSO): δ 8.64 (s, 1H), 8.53 (s, 1H).

### Step 5: 3-(Trifluoromethyl)imidazo[5,1-b][1,3]thiazol-7-amine Hydrochloride (5)

7-Nitro-3-(trifluoromethyl)imidazo[5,1-b][1,3]thiazole (800 mg, 3.4 mmol) and 10 wt % palladium on carbon (360 mg, 0.34 mmol) were placed in a parr shaker vial that was covered with parafilm. Argon was introduced to the vial, and ethyl acetate (17 ml) was introduced via syringe. The vial was taken to the parr shaker and evacuated then backfilled with nitrogen 3 times, then evacuated and backfilled with hydrogen 3 times. The reaction was shaken under 50 psi hydrogen for 8 hours. It was then filtered through a celite plug, eluting with ethyl acetate. 2 M HCl (1.8 ml, 3.54 mmol) in diethyl ether was then slowly added to the filtrate and a white precipitate immediately formed. The slurry was stirred for 5 minutes at room temperature. The solids were collected by filtration and dried under vacuum overnight to afford the title compound. LRMS (APCI) calc'd for C₆H₄F₃N₃S [M+H]⁺: 208.0, Found: 207.8. ¹H NMR (600 MHz, d6-DMSO): δ 10.25 (broad s, 2H), 8.51 (s, 1H), 8.30 (s, 1H).

### Step 6: 2-[4-(2-Oxo-1,3-oxazolidin-3-yl)phenyl]-N-[3-(trifluoromethyl)imidazo[5,1-b][1,3]thiazol-7-yl]acetamide (6)

3-(Trifluoromethyl)imidazo[5,1-b][1,3]thiazol-7-amine hydrochloride (150 mg, 0.62 mmol), [4-(2-oxo-1,3-oxazolidin-3-yl)phenyl]acetic acid (160 mg, 0.74 mmol), EDC (180 mg, 0.92 mmol), and HOBT (140 mg, 0.92 mmol) were placed in a vial and taken up in dichloromethane (6.2 ml). Triethylamine (0.26 ml, 1.85 mmol) was then added and the reaction was stirred at room temperature for 2.5 hours. The resulting solids were collected by filtration to afford the title compound. LRMS (APCI) calc'd for C₁₇H₁₃F₃N₄O₃S [M+H]⁺: 411.1, Found: 410.7. ¹H NMR (600 MHz, d6-DMSO): δ 10.98 (s, 1H), 8.10 (s, 1H), 8.05 (s, 1H), 7.50 (d, 2H), 7.31 (d, 2H), 4.41 (t, 2H), 4.03 (t, 2H), 3.59 (s, 2H).

Additional analogues were prepared using procedures similar to those described in the above example.

TABLE 1

| Example | Structure | Name | Mass ion |
|---|---|---|---|
| 2 | 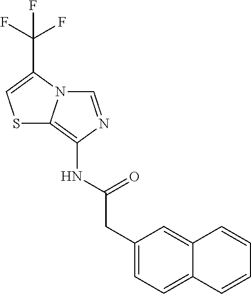 [SMILES: O=C(Cc1ccc2ccccc2c1)Nc1ncn2c(C(F)(F)F)csc12] | 2-(2-naphthyl)- N-[3- (trifluoromethyl) imidazo[5,1- b][1,3]thiazol- 7-yl]acetamide | Calc'd 376.1, found 376.0 |
| 3 | 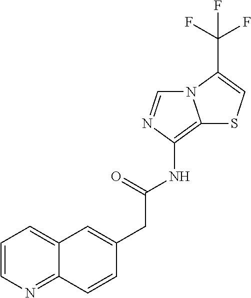 [SMILES: O=C(Cc1ccc2ncccc2c1)Nc1ncn2c(C(F)(F)F)csc12] | 2-quinolin-6-yl- N-[3- (trifluoromethyl) imidazo[5,1- b][1,3]thiazol- 7-yl]acetamide | Calc'd 377.1, found 377.0 |
| 4 | 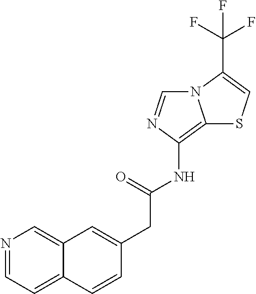 [SMILES: O=C(Cc1ccc2ccncc2c1)Nc1ncn2c(C(F)(F)F)csc12] | 2-isoquinolin-7- yl-N-[3- (trifluoromethyl) imidazo[5,1- b][1,3]thiazol- 7-yl]acetamide | Calc'd 377.1, found 377.0 |
| 5 | 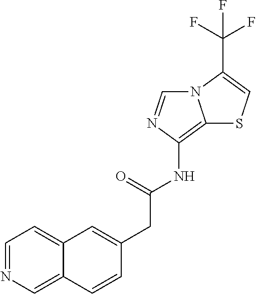 [SMILES: O=C(Cc1ccc2cnccc2c1)Nc1ncn2c(C(F)(F)F)csc12] | 2-isoquinolin-6- yl-N-[3- (trifluoromethyl) imidazo[5,1- b][1,3]thiazol- 7-yl]acetamide | Calc'd 377.1, found 377.0 |
| 6 | 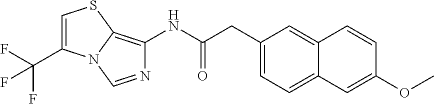 [SMILES: COc1ccc2cc(CC(=O)Nc3ncn4c(C(F)(F)F)csc34)ccc2c1] | 2-(6-methoxy- 2-naphthyl)-N- [3- (trifluoromethyl) imidazo[5,1- b][1,3]thiazol- 7-yl]acetamide | Calc'd 406.1, found 406.0 |
| 7 | 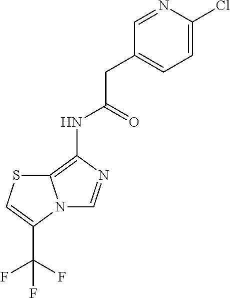 [SMILES: O=C(Cc1ccc(Cl)nc1)Nc1ncn2c(C(F)(F)F)csc12] | 2-(6- chloropyridin-3- yl)-N-[3- (trifluoromethyl) imidazo[5,1- b][1,3]thiazol- 7-yl]acetamide | Calc'd 361.0, found 360.9 |
| 8 | 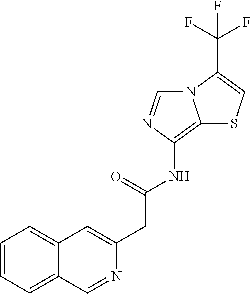 [SMILES: O=C(Cc1cc2ccccc2cn1)Nc1ncn2c(C(F)(F)F)csc12] | 2-isoquinolin-3- yl-N-[3- (trifluoromethyl) imidazo[5,1- b][1,3]thiazol- 7-yl]acetamide | Calc'd 377.1, found 377.0 |
| 9 | 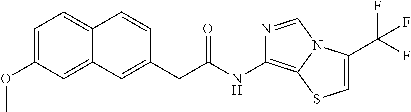 [SMILES: COc1ccc2ccc(CC(=O)Nc3ncn4c(C(F)(F)F)csc34)cc2c1] | 2-(7-methoxy- 2-naphthyl)-N- [3- (trifluoromethyl) imidazo[5,1- b][1,3]thiazol- 7-yl]acetamide | Calc'd 406.1, found 406.0 |
| 10 | 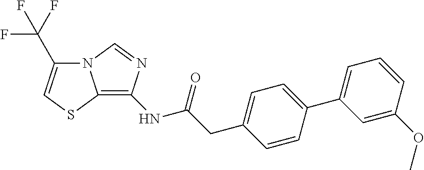 [SMILES: COc1cccc(-c2ccc(CC(=O)Nc3ncn4c(C(F)(F)F)csc34)cc2)c1] | 2-(3′- methoxybiphenyl- 4-yl)-N-[3- (trifluoromethyl) imidazo[5,1- b][1,3]thiazol- 7-yl]acetamide | Calc'd 432.1, found 432.0 |
| 11 | 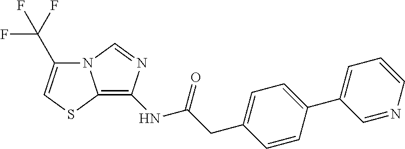 [SMILES: O=C(Cc1ccc(-c2cccnc2)cc1)Nc1ncn2c(C(F)(F)F)csc12] | 2-(4-pyridin-3- ylphenyl)-N-[3- (trifluoromethyl) imidazo[5,1- b][1,3]thiazol- 7-yl]acetamide | Calc'd 403.1, found 403.0 |
| 12 | 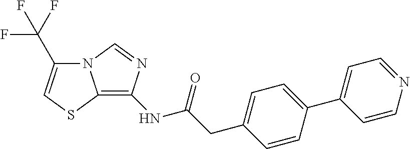 [SMILES: O=C(Cc1ccc(-c2ccncc2)cc1)Nc1ncn2c(C(F)(F)F)csc12] | 2-(4-pyridin-4- ylphenyl)-N-[3- (trifluoromethyl) imidazo[5,1- b][1,3]thiazol- 7-yl]acetamide | Calc'd 403.1, found 403.0 |
| 13 | 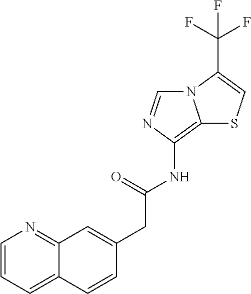 [SMILES: O=C(Cc1ccc2cccnc2c1)Nc1ncn2c(C(F)(F)F)csc12] | 2-quinolin-7-yl- N-(3- (trifluoromethyl) imidazo[5,1- b][1,3]thiazol- 7-yl]acetamide | Calc'd 377.1, found 377.0 |
| 14 | 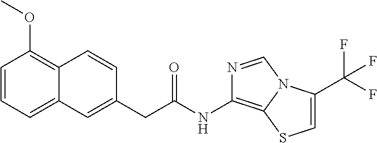 [SMILES: COc1cccc2cc(CC(=O)Nc3ncn4c(C(F)(F)F)csc34)ccc12] | 2-(5-methoxy- 2-naphthyl)-N- [3- (trifluoromethyl) imidazo[5,1- b][1,3]thiazol- 7-yl]acetamide | Calc'd 406.1, found 406.0 |
| 15 | 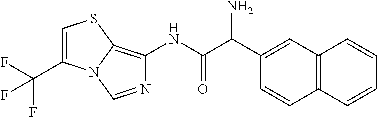 [SMILES: NC(C(=O)Nc1ncn2c(C(F)(F)F)csc12)c1ccc2ccccc2c1] | 2-amino-2-(2- naphthyl)-N-[3- (trifluoromethyl) imidazo[5,1- b][1,3]thiazol- 7-yl]acetamide | Calc'd 391.1, found 391.1 |
| 16 | 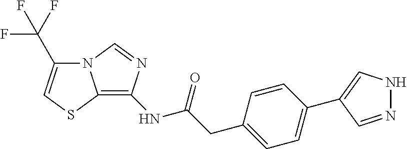 [SMILES: O=C(Cc1ccc(-c2cn[nH]c2)cc1)Nc1ncn2c(C(F)(F)F)csc12] | 2-[4-(1H- pyrazol-4- yl)phenyl]-N- (3- (trifluoromethyl) imidazo[5,1- b][1,3]thiazol- 7-yl]acetamide | Calc'd 392.1 found 392.0 |
| 17 | 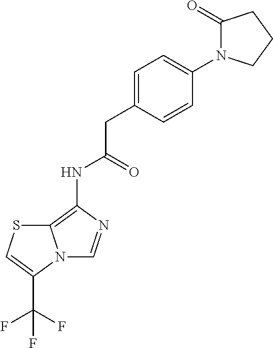 [SMILES: O=C(Cc1ccc(N2CCCC2=O)cc1)Nc1ncn2c(C(F)(F)F)csc12] | 2-[4-(2- oxopyrrolidin- 1-yl)phenyl]-N- [3- (trifluoromethyl) imidazo[5,1- b][1,3]thiazol- 7-yl]acetamide | Calc'd 409.1, found 409.1 |
| 18 | 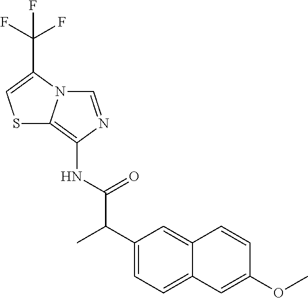 [SMILES: COc1ccc2cc(C(C)C(=O)Nc3ncn4c(C(F)(F)F)csc34)ccc2c1] | 2-(6-methoxy- 2-naphthyl)-N- [3- (trifluoromethyl) imidazo[5,1- b][1,3]thiazol- 7- yl]propanamide | Calc'd 420.1, found 420.1 |
| 19 | 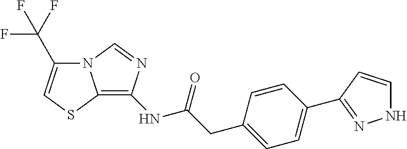 [SMILES: O=C(Cc1ccc(-c2cc[nH]n2)cc1)Nc1ncn2c(C(F)(F)F)csc12] | 2-[4-(1H- pyrazol-3- yl)phenyl]-N- [3- (trifluoromethyl) imidazo[5,1- b][1,3]thiazol- 7-yl]acetamide | Calc'd 392.1 found 392.0 |
| 20 | 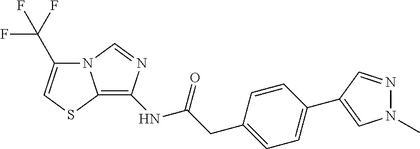 [SMILES: Cn1cc(-c2ccc(CC(=O)Nc3ncn4c(C(F)(F)F)csc34)cc2)cn1] | 2-[4-(1-methyl- 1H-pyrazol-4- yl)phenyl]-N- [3- (trifluoromethyl) imidazo[5,1- b][1,3]thiazol- 7-yl]acetamide | Calc'd 406.1 found 406.0 |
| 21 | 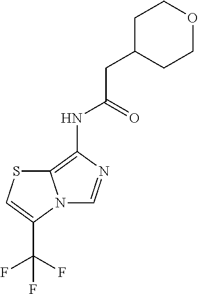 [SMILES: O=C(CC1CCOCC1)Nc1ncn2c(C(F)(F)F)csc12] | 2-(tetrahydro- 2H-pyran-4-yl)- N-[3- (trifluoromethyl) imidazo[5,1- b][1,3]thiazol- 7-yl]acetamide | Calc'd 334.1 found 334.0 |
| 22 | 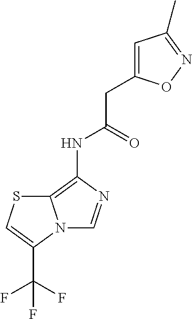 [SMILES: Cc1cc(CC(=O)Nc2ncn3c(C(F)(F)F)csc23)on1] | 2-(3- methylisoxazol- 5-yl)-N-[3- (trifluoromethyl) imidazo[5,1- b][1,3]thiazol- 7-yl]acetamide | Calc'd 331.0 found 331.0 |
| 23 | 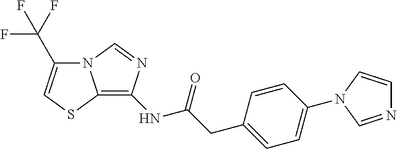 [SMILES: O=C(Cc1ccc(-n2ccnc2)cc1)Nc1ncn2c(C(F)(F)F)csc12] | 2-[4-(1H- imidazol-1- yl)phenyl]-N- [3- (trifluoromethyl) imidazo[5,1- b][1,3]thiazol- 7-yl]acetamide | Calc'd 392.1 found 392.0 |
| 24 | 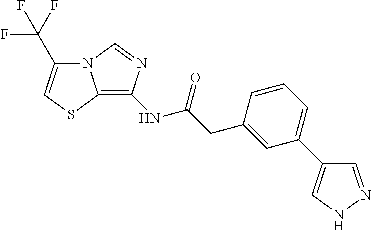 [SMILES: O=C(Cc1cccc(-c2cn[nH]c2)c1)Nc1ncn2c(C(F)(F)F)csc12] | 2-[3-(1H- pyrazol-4- yl)phenyl]-N- [3- (trifluoromethyl) imidazo[5,1- b][1,3]thiazol- 7-yl]acetamide | Calc'd 392.1 found 392.0 |
| 25 | 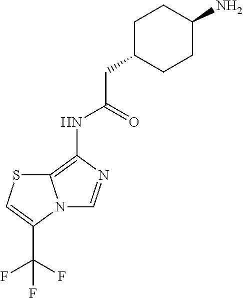 [SMILES: N[C@H]1CC[C@H](CC(=O)Nc2ncn3c(C(F)(F)F)csc23)CC1] | 2-(trans-4- amino- cyclohexyl)- N-[3- (trifluoromethyl) imidazo[5,1- b][1,3]thiazol- 7-yl]acetamide | Calc'd 347.1 found 347.1 |
| 26 | 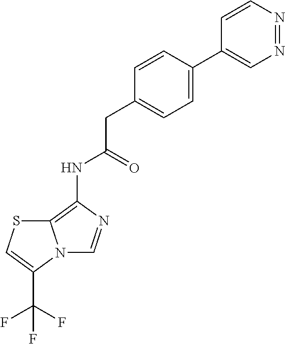 [SMILES: O=C(Cc1ccc(-c2ccnnc2)cc1)Nc1ncn2c(C(F)(F)F)csc12] | 2-(4-pyridazin- 4-ylphenyl)-N- [3- (trifluoromethyl) imidazo[5,1- b][1,3]thiazol- 7-yl]acetamide | Calc'd 404.1 found 404.0 |
| 27 | 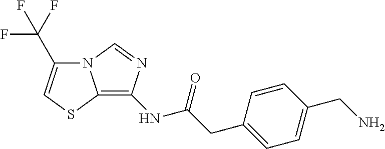 [SMILES: NCc1ccc(CC(=O)Nc2ncn3c(C(F)(F)F)csc23)cc1] | 2-[4- (aminomethyl) phenyl]-N-[3- (trifluoromethyl) imidazo[5,1- b][1,3]thiazol- 7-yl]acetamide | Calc'd 355.1 found 354.9 |
| 28 | 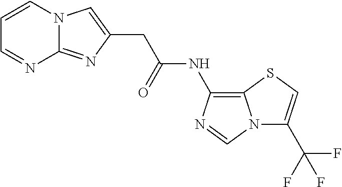 [SMILES: O=C(Cc1cn2cccnc2n1)Nc1ncn2c(C(F)(F)F)csc12] | 2-imidazo[1,2- a]pyrimidin-2- yl-N-[3- (trifluoromethyl) imidazo[5,1- b][1,3]thiazol- 7-yl]acetamide | Calc'd 367.1, found 366.9 |
| 29 | 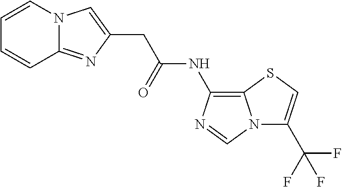 [SMILES: O=C(Cc1cn2ccccc2n1)Nc1ncn2c(C(F)(F)F)csc12] | 2-imidazo[1,2- a]pyridin-2-yl- N-[3- (trifluoromethyl) imidazo[5,1- b][1,3]thiazol- 7-yl]acetamide | Calc'd 366.1, found 365.9 |
| 30 | 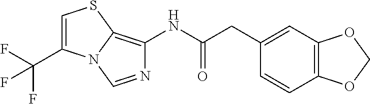 [SMILES: O=C(Cc1ccc2c(c1)OCO2)Nc1ncn2c(C(F)(F)F)csc12] | 2-(1,3- benzodioxol-5- yl)-N-[3- (trifluoromethyl) imidazo[5,1- b][1,3]thiazol- 7-yl]acetamide | Calc'd 370.0, found 369.9 |
| 31 | 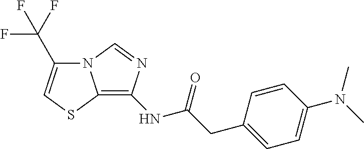 [SMILES: CN(C)c1ccc(CC(=O)Nc2ncn3c(C(F)(F)F)csc23)cc1] | 2-[4- (dimethylamino) phenyl]-N-[3- (trifluoromethyl) imidazo[5,1- b][1,3]thiazol- 7-yl]acetamide | Calc'd 369.1, found 368.9 |
| 32 | 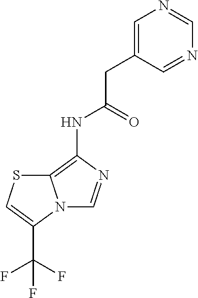 [SMILES: O=C(Cc1cncnc1)Nc1ncn2c(C(F)(F)F)csc12] | 2-pyrimidin-5- yl-N-[3- (trifluoromethyl) imidazo[5,1- b][1,3]thiazol- 7-yl]acetamide | Calc'd 328.0, found 327.9 |
| 33 | 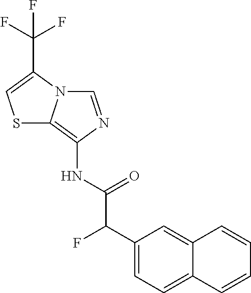 [SMILES: O=C(Nc1ncn2c(C(F)(F)F)csc12)C(F)c1ccc2ccccc2c1] | 2-fluoro-2-(2- naphthyl)-N-[3- (trifluoromethyl) imidazo[5,1- b][1,3]thiazol- 7-yl]acetamide | Calc'd 394.1, found 393.9 |
| 34 | 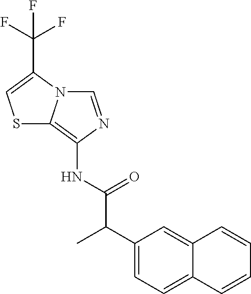 [SMILES: CC(C(=O)Nc1ncn2c(C(F)(F)F)csc12)c1ccc2ccccc2c1] | 2-(2-naphthyl)- N-[3- (trifluoromethyl) imidazo[5,1- b][1,3]thiazol- 7- yl]propanamide | Calc'd 390.1, found 389.9 |
| 35 | 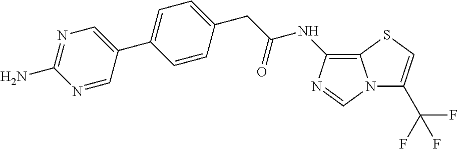 [SMILES: Nc1ncc(-c2ccc(CC(=O)Nc3ncn4c(C(F)(F)F)csc34)cc2)cn1] | 2-[4-(2- aminopyrimidin- 5-yl)phenyl]- N-[3- (trifluoromethyl) imidazo[5,1- b][1,3]thiazol- 7-yl]acetamide | Calc'd 419.1, found 418.9 |
| 36 | 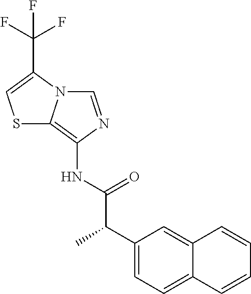 [SMILES: C[C@H](C(=O)Nc1ncn2c(C(F)(F)F)csc12)c1ccc2ccccc2c1] | (2S)-2-(2- naphthyl)-N-[3- (trifluoromethyl) imidazo[5,1- b][1,3]thiazol- 7- yl]propanamide | Calc'd 390.1, found 389.9 |
| 37 | 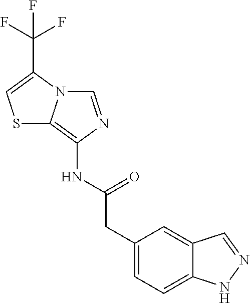 [SMILES: O=C(Cc1ccc2[nH]ncc2c1)Nc1ncn2c(C(F)(F)F)csc12] | 2-(1H-indazol- 5-yl)-N-[3- (trifluoromethyl) imidazo[5,1- b][1,3]thiazol- 7-yl]acetamide | Calc'd 366.1 found 365.9 |
| 38 | 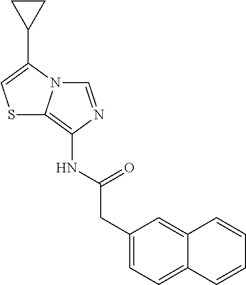 [SMILES: O=C(Cc1ccc2ccccc2c1)Nc1ncn2c(C3CC3)csc12] | N-(3- cyclopropyl- imidazo[5,1- b][1,3]thiazol- 7-yl)-2-(2- naphthyl) acetamide | Calc'd 348.1, found 348.1 |
| 39 | 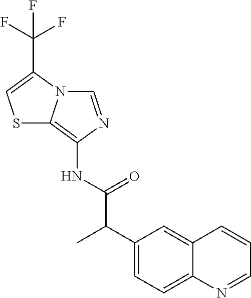 [SMILES: CC(C(=O)Nc1ncn2c(C(F)(F)F)csc12)c1ccc2ncccc2c1] | 2-quinolin-6-yl- N-[3- (trifluoromethyl) imidazo[5,1- b][1,3]thiazol- 7- yl]propanamide | Calc'd 391.1, found 391.1 |
| 40 | 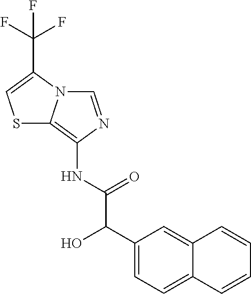 [SMILES: O=C(Nc1ncn2c(C(F)(F)F)csc12)C(O)c1ccc2ccccc2c1] | 2-hydroxy-2-(2- naphthyl)-N-[3- (trifluoromethyl) imidazo[5,1- b][1,3]thiazol- 7-yl]acetamide | Calc'd 392.1, found 392.0 |
| 41 | 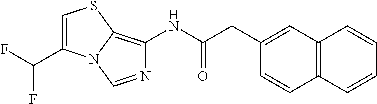 [SMILES: O=C(Cc1ccc2ccccc2c1)Nc1ncn2c(C(F)F)csc12] | N-[3- (difluoromethyl) imidazo[5,1- b][1,3]thiazol- 7-yl]-2-(2- naphthyl) acetamide | Calc'd 358.1, found 358.0 |
| 42 | 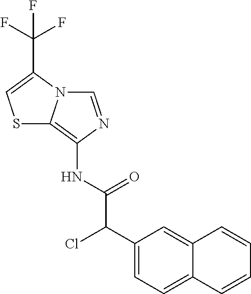 [SMILES: O=C(Nc1ncn2c(C(F)(F)F)csc12)C(Cl)c1ccc2ccccc2c1] | 2-chloro-2-(2- naphthyl)-N-[3- (trifluoromethyl) imidazo[5,1- b][1,3]thiazol- 7-yl]acetamide | Calc'd 410.0, found 410.0 |
| 43 | 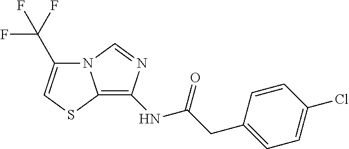 [SMILES: O=C(Cc1ccc(Cl)cc1)Nc1ncn2c(C(F)(F)F)csc12] | 2-(4- chlorophenyl)- N-[3- (trifluoromethyl) imidazo[5,1- b][1,3]thiazol- 7-yl]acetamide | Calc'd 360.0, found 359.7 |
| 44 | 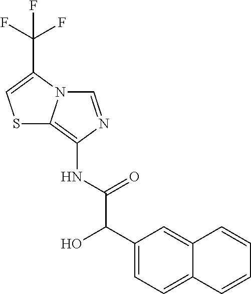 [SMILES: O=C(Nc1ncn2c(C(F)(F)F)csc12)C(O)c1ccc2ccccc2c1] | (R or S)-2- hydroxy-2-(2- naphthyl)-N-[3- (trifluoromethyl) imidazo[5,1- b][1,3]thiazol- 7-yl]acetamide | Calc'd 392.1, found 391.7 |
| 45 | 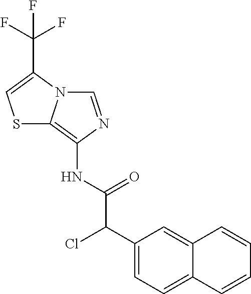 [SMILES: O=C(Nc1ncn2c(C(F)(F)F)csc12)C(Cl)c1ccc2ccccc2c1] | (R or S)-2- chloro-2-(2- naphthyl)-N-[3- (trifluoromethyl) imidazo[5,1- b][1,3]thiazol- 7-yl]acetamide | Calc'd 410.0, found 409.7 |
| 46 | 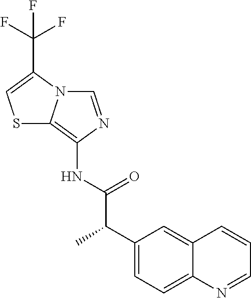 [SMILES: C[C@H](C(=O)Nc1ncn2c(C(F)(F)F)csc12)c1ccc2ncccc2c1] | (2S)-2-quinolin- 6-yl-N-[3- (trifluoromethyl) imidazo[5,1- b][1,3]thiazol- 7- yl]propanamide | Calc'd 391.1, found 390.7 |
| 47 | 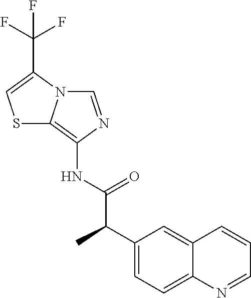 [SMILES: C[C@@H](C(=O)Nc1ncn2c(C(F)(F)F)csc12)c1ccc2ncccc2c1] | (2R)-2- quinolin-6-yl- N-[3- (trifluoromethyl) imidazo[5,1- b][1,3]thiazol- 7- yl]propanamide | Calc'd 391.1, found 390.8 |
| 48 | 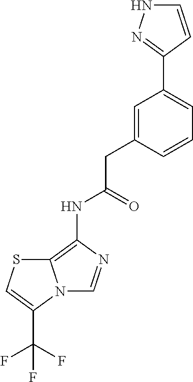 [SMILES: O=C(Cc1cccc(-c2cc[nH]n2)c1)Nc1ncn2c(C(F)(F)F)csc12] | 2-[3-(1H- pyrazol-3- yl)phenyl]-N- [3- (trifluoromethyl) imidazo[5,1- b][1,3]thiazol- 7-yl]acetamide | Calc'd 392.1 found 392.9 |
| 49 | 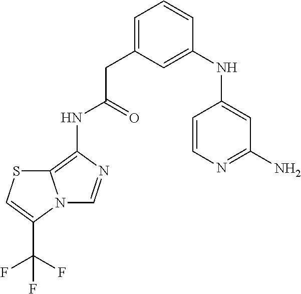 [SMILES: Nc1cc(Nc2cccc(CC(=O)Nc3ncn4c(C(F)(F)F)csc34)c2)ccn1] | 2-{3-[(2- aminopyridin-4- yl)amino]phenyl}- N-[3- (trifluoromethyl) imidazo[5,1- b][1,3]thiazol- 7-yl]acetamide | Calc'd 433.1, found 433.1 |
| 50 | 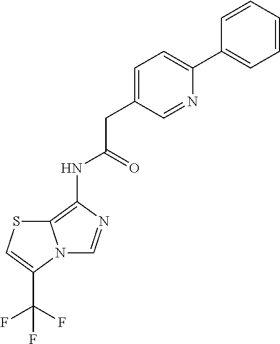 [SMILES: O=C(Cc1ccc(-c2ccccc2)nc1)Nc1ncn2c(C(F)(F)F)csc12] | 2-(6- phenylpyridin- 3-yl)-N-[3- (trifluoromethyl) imidazo[5,1- b][1,3]thiazol- 7-yl]acetamide | Calc'd 403.1 found 402.9 |
| 51 | 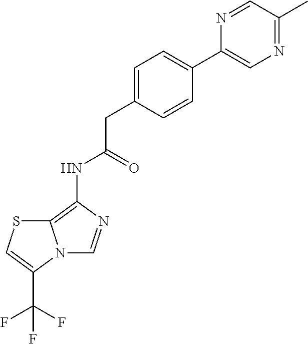 [SMILES: Cc1cnc(-c2ccc(CC(=O)Nc3ncn4c(C(F)(F)F)csc34)cc2)cn1] | 2-[4-(5- methylpyrazin- 2-yl)phenyl]-N- [3- (trifluoromethyl) imidazo[5,1- b][1,3]thiazol- 7-yl]acetamide | Calc'd 418.1 found 417.9 |
| 52 | 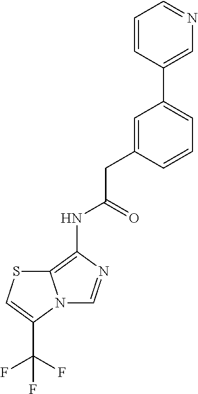 [SMILES: O=C(Cc1cccc(-c2cccnc2)c1)Nc1ncn2c(C(F)(F)F)csc12] | 2-(3-pyridin-3- ylphenyl)-N-[3- (trifluoromethyl) imidazo[5,1- b][1,3]thiazol- 7-yl]acetamide | Calc'd 403.1 found 402.9 |
| 53 | 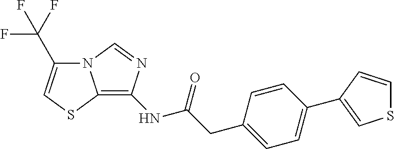 [SMILES: O=C(Cc1ccc(-c2ccsc2)cc1)Nc1ncn2c(C(F)(F)F)csc12] | 2-[4-(3- thienyl)phenyl]- N-[3- (trifluoromethyl) imidazo[5,1- b][1,3]thiazol- 7-yl]acetamide | Calc'd 408.0, found 407.7 |
| 54 | 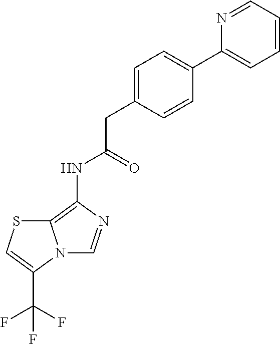 [SMILES: O=C(Cc1ccc(-c2ccccn2)cc1)Nc1ncn2c(C(F)(F)F)csc12] | 2-(4-pyridin-2- ylphenyl)-N-[3- (trifluoromethyl) imidazo[5,1- b][1,3]thiazol- 7-yl]acetamide | Calc'd 403.1 found 402.9 |
| 55 | 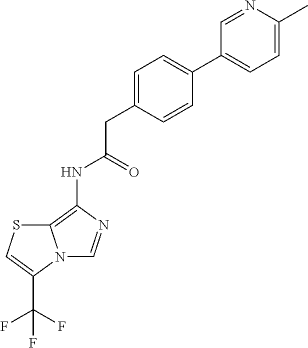 [SMILES: Cc1ccc(-c2ccc(CC(=O)Nc3ncn4c(C(F)(F)F)csc34)cc2)cn1] | 2-[4-(6- methylpyridin- 3-yl)phenyl]-N- [3- (trifluoromethyl) imidazo[5,1- b][1,3]thiazol- 7-yl]acetamide | Calc'd 417.1 found 416.9 |
| 56 | 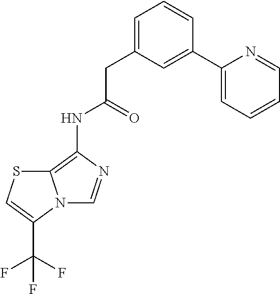 [SMILES: O=C(Cc1cccc(-c2ccccn2)c1)Nc1ncn2c(C(F)(F)F)csc12] | 2-(3-pyridin-2- ylphenyl)-N-[3- (trifluoromethyl) imidazo[5,1- b][1,3]thiazol- 7-yl]acetamide | Calc'd 403.1 found 402.9 |
| 57 | 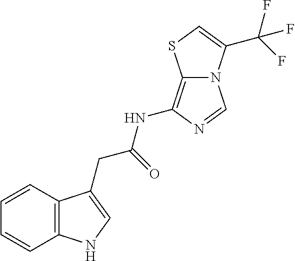 [SMILES: O=C(Cc1c[nH]c2ccccc12)Nc1ncn2c(C(F)(F)F)csc12] | 2-(1H-indol-3- yl)-N-[3- (trifluoromethyl) imidazo[5,1- b][1,3]thiazol- 7-yl]acetamide | Calc'd 365.1 found 364.9 |
| 58 | 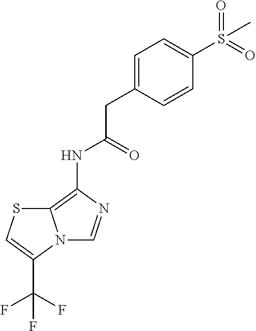 [SMILES: CS(=O)(=O)c1ccc(CC(=O)Nc2ncn3c(C(F)(F)F)csc23)cc1] | 2-[4- (methylsulfonyl) phenyl]-N-[3- (trifluoromethyl) imidazo[5,1- b][1,3]thiazol- 7-yl]acetamide | Calc'd 404.0 found 403.8 |
| 59 | 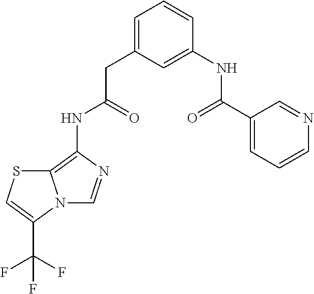 [SMILES: O=C(Cc1cccc(NC(=O)c2cccnc2)c1)Nc1ncn2c(C(F)(F)F)csc12] | N-[3-(2-oxo-2- {[3- (trifluoromethyl) imidazo[5,1- b][1,3]thiazol- 7- yl]amino}ethyl) phenyl] nicotinamide | Calc'd 446.1, found 445.7 |
| 60 | 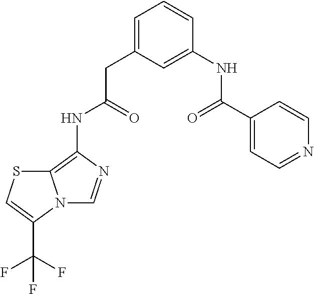 [SMILES: O=C(Cc1cccc(NC(=O)c2ccncc2)c1)Nc1ncn2c(C(F)(F)F)csc12] | N-[3-(2-oxo-2- {[3- (trifluoromethyl) imidazo[5,1- b][1,3]thiazol- 7- yl]amino}ethyl) phenyl] isonicotinamide | Calc'd 446.1, found 445.7 |
| 61 | 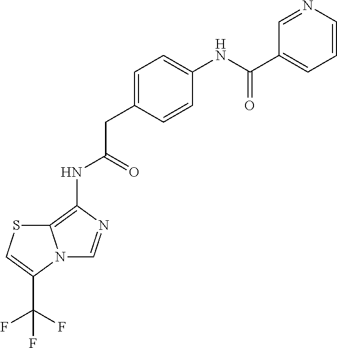 [SMILES: O=C(Cc1ccc(NC(=O)c2cccnc2)cc1)Nc1ncn2c(C(F)(F)F)csc12] | N-[4-(2-oxo-2- {[3- (trifluoromethyl) imidazo[5,1- b][1,3]thiazol- 7- yl]amino}ethyl) phenyl] nicotinamide | Calc'd 446.1, found 445.7 |
| 62 | 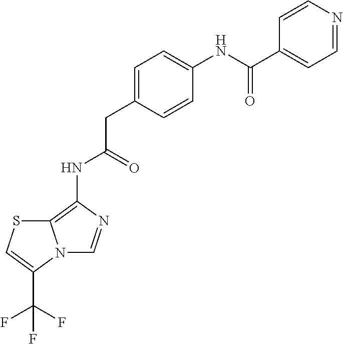 [SMILES: O=C(Cc1ccc(NC(=O)c2ccncc2)cc1)Nc1ncn2c(C(F)(F)F)csc12] | N-[4-(2-oxo-2- {[3- (trifluoromethyl) imidazo[5,1- b][1,3]thiazol- 7- yl]amino}ethyl) phenyl] isonicotinamide | Calc'd 446.1, found 445.7 |
| 63 | 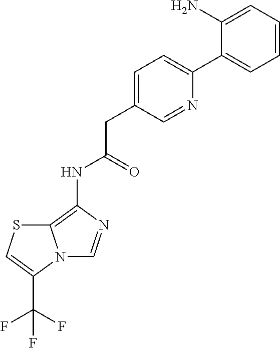 [SMILES: Nc1ccccc1-c1ccc(CC(=O)Nc2ncn3c(C(F)(F)F)csc23)cn1] | 2-[6-(2- aminophenyl) pyridin-3-yl]-N- [3- (trifluoromethyl) imidazo[5,1- b][1,3]thiazol- 7-yl]acetamide | Calc'd 418.1, found 417.7 |
| 64 | 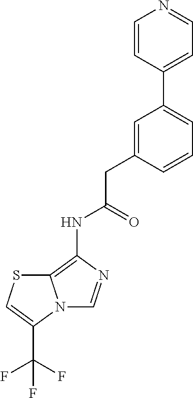 [SMILES: O=C(Cc1cccc(-c2ccncc2)c1)Nc1ncn2c(C(F)(F)F)csc12] | 2-(3-pyridin-4- ylphenyl)-N-[3- (trifluoromethyl) imidazo[5,1- b][1,3]thiazol- 7-yl]acetamide | Calc'd 403.1 found 402.9 |
| 65 | 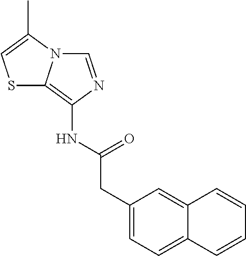 [SMILES: Cc1csc2c(NC(=O)Cc3ccc4ccccc4c3)ncn12] | N-(3- methylimidazo [5,1- b][1,3]thiazol- 7-yl)-2-(2- naphthyl) acetamide | Calc'd 322.1, found 321.8 |
| 66 | 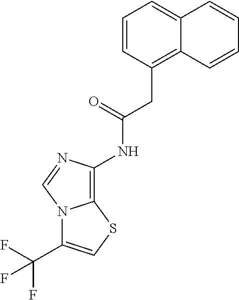 [SMILES: O=C(Cc1cccc2ccccc12)Nc1ncn2c(C(F)(F)F)csc12] | 2-(1-naphthyl)- N-[3- (trifluoromethyl) imidazo[5,1- b][1,3]thiazol- 7-yl]acetamide | Calc'd 376.1, found 375.9 |
| 67 | 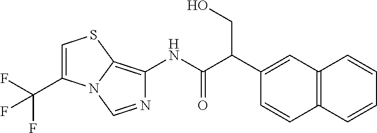 [SMILES: O=C(Nc1ncn2c(C(F)(F)F)csc12)C(CO)c1ccc2ccccc2c1] | 3-hydroxy-2-(2- naphthyl)-N-[3- (trifluoromethyl) imidazo[5,1- b][1,3]thiazol- 7- yl]propanamide | Calc'd 406.1, found 405.7 |
| 68 | 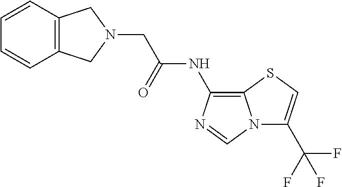 [SMILES: O=C(CN1Cc2ccccc2C1)Nc1ncn2c(C(F)(F)F)csc12] | 2-(1,3-dihydro- 2H-isoindol-2- yl)-N-[3- (trifluoromethyl) imidazo[5,1- b][1,3]thiazol- 7-yl]acetamide | Calc'd 367.1, found 366.7 |
| 69 | 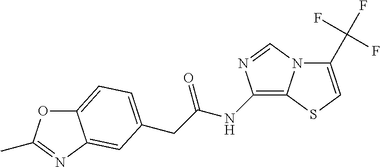 [SMILES: Cc1nc2cc(CC(=O)Nc3ncn4c(C(F)(F)F)csc34)ccc2o1] | 2-(2-methyl- 1,3-benzoxazol- 5-yl)-N-[3- (trifluoromethyl) imidazo[5,1- b][1,3]thiazol- 7-yl]acetamide | Calc'd 381.1, found 380.9 |
| 70 | 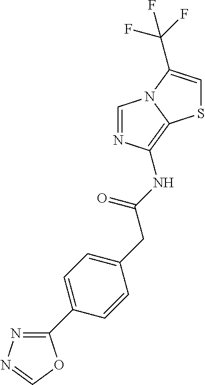 [SMILES: O=C(Cc1ccc(-c2nnco2)cc1)Nc1ncn2c(C(F)(F)F)csc12] | 2-[4-(1,3,4- oxadiazol-2- yl)phenyl]-N- [3- (trifluoromethyl) imidazo[5,1- b][1,3]thiazol- 7-yl]acetamide | Calc'd 394.1, found 393.9 |
| 71 | 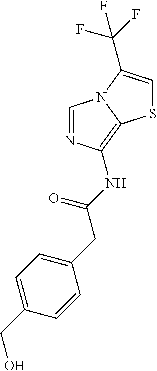 [SMILES: O=C(Cc1ccc(CO)cc1)Nc1ncn2c(C(F)(F)F)csc12] | 2-[4- (hydroxymethyl) phenyl]-N-[3- (trifluoromethyl) imidazo[5,1- b][1,3]thiazol- 7-yl]acetamide | Calc'd 356.1, found 355.7 |
| 72 | 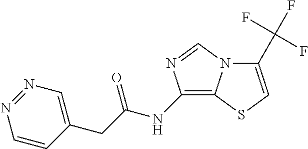 [SMILES: O=C(Cc1ccnnc1)Nc1ncn2c(C(F)(F)F)csc12] | 2-pyridazin-4- yl-N-[3- (trifluoromethyl) imidazo[5,1- b][1,3]thiazol- 7-yl]acetamide | Calc'd 328.0, found 327.9 |
| 73 | 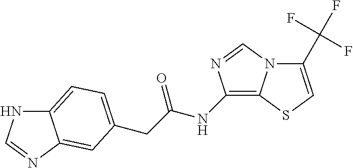 [SMILES: O=C(Cc1ccc2[nH]cnc2c1)Nc1ncn2c(C(F)(F)F)csc12] | 2-(1H- benzimidazol-5- yl)-N-[3- (trifluoromethyl) imidazo[5,1- b][1,3]thiazol- 7-yl]acetamide | Calc'd 366.1, found 365.9 |
| 74 | 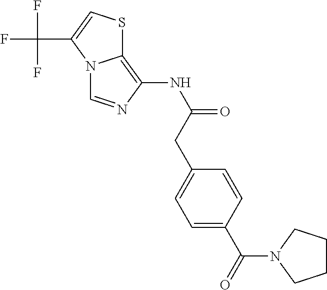 [SMILES: O=C(Cc1ccc(C(=O)N2CCCC2)cc1)Nc1ncn2c(C(F)(F)F)csc12] | 2-[4- (pyrrolidin-1- ylcarbonyl) phenyl]-N-[3- (trifluoromethyl) imidazo[5,1- b][1,3]thiazol- 7-yl]acetamide | Calc'd 423.1, found 422.9 |
| 75 | 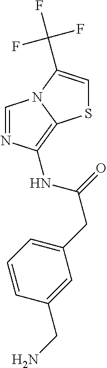 [SMILES: NCc1cccc(CC(=O)Nc2ncn3c(C(F)(F)F)csc23)c1] | 2-[3- (aminomethyl) phenyl]-N-[3- (trifluoromethyl) imidazo[5,1- b][1,3]thiazol- 7-yl]acetamide | Calc'd 355.1, found 354.9 |
| 76 | 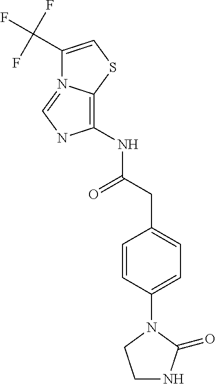 | 2-[4-(2- oxoimidazolidin- 1-yl)phenyl]- N-[3- (trifluoromethyl) imidazo[5,1- b][1,3]thiazol- 7-yl]acetamide | Calc'd 410.1, found 409.9 |
| 77 | 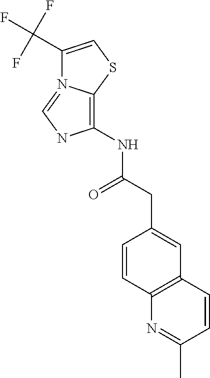 | 2-(2- methylquinolin- 6-yl)-N-[3- (trifluoromethyl) imidazo[5,1- b][1,3]thiazol- 7-yl]acetamide | Calc'd 391.1, found 390.9 |
| 78 | 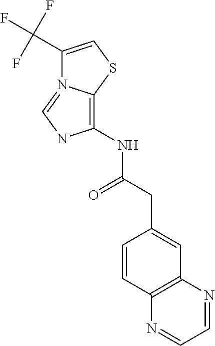 | 2-quinoxalin-6- yl-N-[3- (trifluoromethyl) imidazo[5,1- b][1,3]thiazol- 7-yl]acetamide | Calc'd 378.1, found 377.9 |
| 79 | 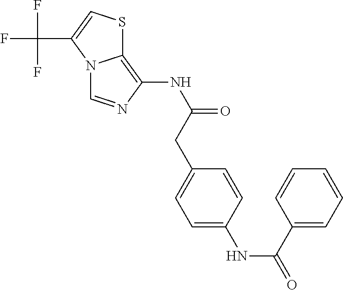 [SMILES: O=C(Cc1ccc(NC(=O)c2ccccc2)cc1)Nc1ncn2c(C(F)(F)F)csc12] | N-[4-(2-oxo-2- {[3- (trifluoromethyl) imidazo[5,1- b][1,3]thiazol- 7- yl)amino}ethyl) phenyl] benzamide | Calc'd 445.1, found 444.7 |
| 80 | 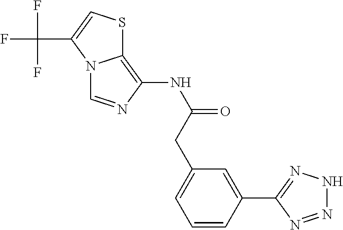 [SMILES: O=C(Cc1cccc(-c2nn[nH]n2)c1)Nc1ncn2c(C(F)(F)F)csc12] | 2-[3-(2H- tetrazol-5- yl)phenyl]-N- [3- (trifluoromethyl) imidazo[5,1- b][1,3]thiazol- 7-yl]acetamide | Calc'd 394.1 found 393.7 |
| 81 | 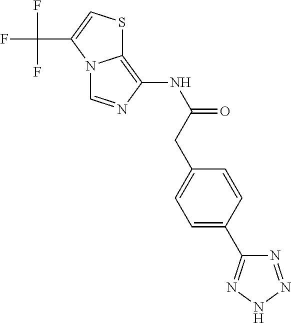 [SMILES: O=C(Cc1ccc(-c2nn[nH]n2)cc1)Nc1ncn2c(C(F)(F)F)csc12] | 2-[4-(2H- tetrazol-5- yl)phenyl]-N- [3- (trifluoromethyl) imidazo[5,1- b][1,3]thiazol- 7-yl]acetamide | Calc'd 394.1, found 393.7 |

### Example 82

### N-imidazo[5,1-b][1,3]thiazol-7-yl-2-(2-naphthyl)acetamide (7)

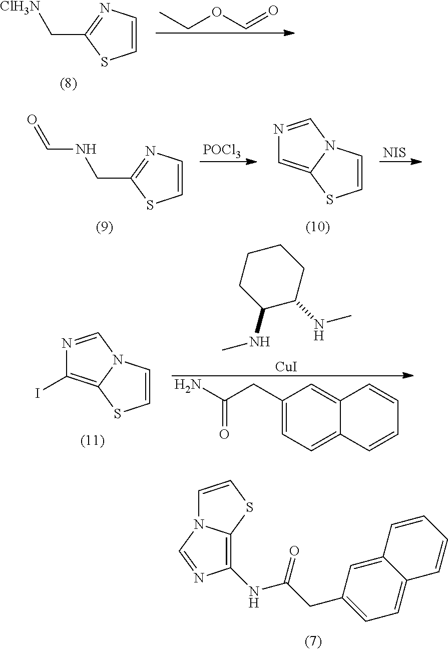

### Step 1: N-(1,3-thiazol-2-ylmethyl)formamide (9)

1-(1,3-Thiazol-2-yl)methanamine hydrochloride (1.5 g, 10 mmol) was taken up in ethyl formate (24 ml, 300 mmol) and N,N-diisopropylethylamine (7 ml, 40 mmol) was added. The mixture was heated at 60° C. overnight. The resulting solution was concentrated under reduced pressure and purified via silica gel chromatography (0-15% methanol in ethyl acetate) to afford the title compound contaminated with ˜12% of bisformylated material. The mixture was not purified further.

### Step 2: Imidazo[5,1-b][1,3]thiazole (10)

N-(1,3-thiazol-2-ylmethyl)formamide (1.2 g, 8.4 mmol) was taken up in phosphorous oxychloride (13 ml, 140 mmol). The mixture was heated to 60° C. for 3.5 hours. The reaction was cooled to room temperature, poured over ice, and then neutralized with 6N aqueous sodium hydroxide. The resulting mixture was extracted with ethyl acetate (3×). The combined organics were dried over magnesium sulfate and concentrated under reduced pressure. The resulting residue was purified via silica gel chromatography (0-20% methanol/ethyl acetate) to afford the title compound as a tan solid. LRMS (APCI) calc'd for C₅H₄N₂S [M+H]⁺: 125.0, Found: 125.0.

### Step 3: 7-iodoimidazo[5,1-b][1,3]thiazole (11)

Imidazo[5,1-b][1,3]thiazole (0.20 g, 1.61 mmol) was taken up in N,N-dimethylformamide (8.1 ml) and cooled to 0° C. N-iodosuccinimide (0.36 g, 1.595 mmol) was added. The mixture was stirred at 0° C. for 30 minutes followed by 30 minutes at room temperature. The solution was concentrated to ⅓ its volume, diluted with ethyl acetate and washed with 1.0 N aqueous sodium hydroxide. The organic layer was dried over magnesium sulfate, filtered, and concentrated under reduced pressure. The resulting residue was purified via silica gel chromatography (0-100% ethyl acetate/hexanes to afford the title compound as a white solid.

### Step 4: N-imidazo[5,1-b][1,3]thiazol-7-yl-2-(2-naphthyl)acetamide (12)

2-(Naphthalen-2-yl)acetamide (44 mg, 0.24 mmol), 7-iodoimidazo[5,1-b][1,3]thiazole (50 mg, 0.20 mmol), potassium phosphate, tribasic (85 mg, 0.40 mmol), and copper(I) iodide (7 mg, 0.04 mmol) were added to a sealed tube which was evacuated and backfilled with argon (3×). Fully degassed toluene (0.8 ml) was added followed by 1,2-trans-N,N′-dimethyldiaminocyclohexane (12 μl, 0.07 mmol). The tube was sealed, and placed in an oil bath at 110° C. After 18 hours, the reaction mixture was cooled to room temperature and concentrated under reduced pressure. The resulting residue was purified via silica gel chromatography (0-15% ethyl acetate/methanol) followed by purification by reverse phase HPLC (15-80% acteinitrile/water+0.05% TFA modifier). Desired fractions were poured into saturated aqueous sodium bicarbonate and extracted with ethyl acetate. The organic layer was dried over magnesium sulfate, filtered, and concentrated in vacuo to afford the title compound as an off-white solid. LRMS (APCI) calc'd for C₁₇H₁₃N₃OS [M+H]⁺: 308.1, Found: 307.9. ¹H NMR (500 MHz, d4-methanol): δ 7.92 (s, 1H), 7.84 (m, 4H), 7.58 (d, 1H), 7.47 (m, 3H), 6.96 (d, 1H), 3.86 (s, 2H).

### Example 83

### N-(1H-imidazol-5-ylmethyl)formamide (12)

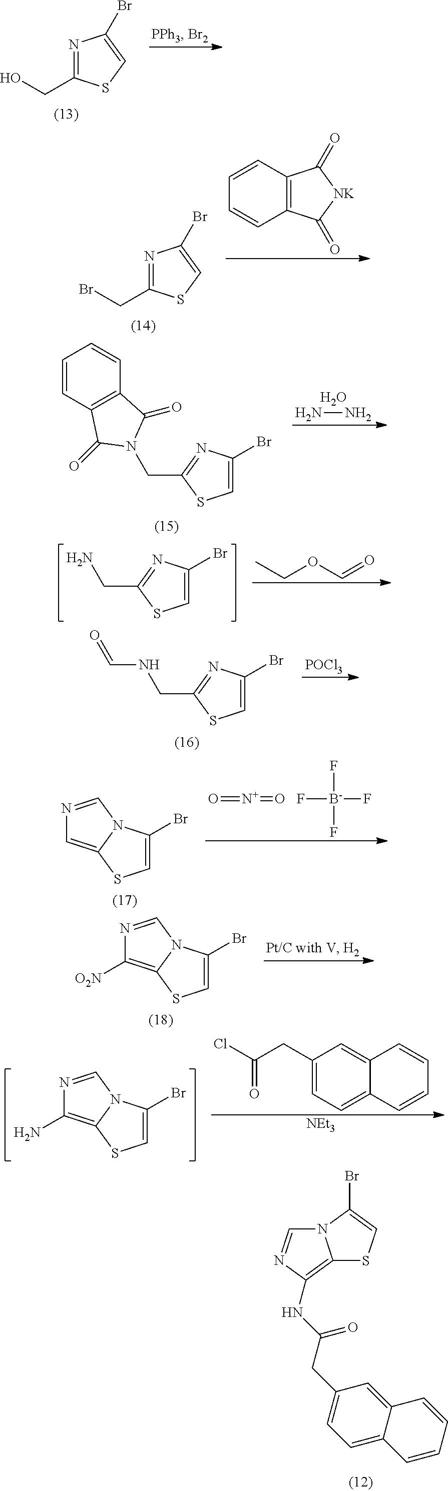

### Step 1: 4-Bromo-2-(bromomethyl)-1,3-thiazole (14)

Triphenylphosphine (8.3 g, 32 mmol) and imidazole (2.4 g, 35 mmol) were taken up in dichloromethane (68 ml). The mixture was cooled to 0° C. and bromine (1.6 ml, 31 mmol) was added. The mixture was stirred for 30 minutes at which time a solution of (4-bromo-1,3-thiazol-2-yl)methanol (4.6 g, 24 mmol) in dichloromethane (34 ml) was added dropwise. The resulting solution was stirred for 30 minutes at 0° C. and then allowed to warm to room temperature over 90 min. The reaction was directly purified via flash chromatography (dry loaded onto 17 g of silica and eluted with 0-30% ethyl acetate/hexanes) to afford the title compound as a light yellow oil. ¹H NMR (500 MHz, d6-DMSO): δ 7.90 (s, 1H), 5.01 (s, 2H).

### Step 2: 2-[(4-Bromo-1,3-thiazol-2-yl)methyl]-1H-isoindole-1,3(2H)-dione (15)

Potassium phthalimide (3.8 g, 21 mmol) was added into a well-stirred solution of 4-bromo-2-(bromomethyl)-1,3-thiazole (5.3 g, 21 mmol) and 18-crown-6 (5.5 g, 21 mmol) in dioxane (100 ml). The mixture was refluxed for 22 hours, cooled to room temperature and filtered. The filtrate was concentrated under reduced pressure to afford the title compound as a light yellow solid. LRMS (APCI) calc'd for C₁₂H₇BrN₂O2S [M+1]⁺, [M+3]⁺: 322.9, 324.9. Found: 322.7, 324.7. ¹H NMR (500 MHz, d6-DMSO): δ 7.92 (s, 2H), 7.88 (s, 2H), 7.80 (s, 1H), 5.09 (s, 2H).

### Step 3: N-[(4-Bromo-1,3-thiazol-2-yl)methyl]formamide (16)

Hydrazine hydrate (2.0 ml, 41 mmol) was added into a well-stirred suspension of 2-[(4-bromo-1,3-thiazol-2-yl)methyl]-1H-isoindole-1,3(2H)-dione (6.6 g, 21 mmol) in boiling ethanol (100 ml). The mixture was refluxed for 24 hours, cooled to room temperature and filtered. The filtrate was concentrated under reduced pressure. The resulting residue was taken up in a mixture of ethyl acetate and dichloromethane. A white precipitate crashed out. The slurry was filtered and the filtrate was concentrated under reduced pressure to afford 1-(4-bromo-1,3-thiazol-2-yl)methanamine as a light yellow oil. This material was taken up in ethyl formate (100 ml, 1.2 mol) and N,N-diisopropylethylamine (3.6 ml, 20 mmol) was added. The mixture was heated at 65° C. overnight, cooled to room temperature and stirred as such for 24 hours. The resulting solution was concentrated under reduced pressure. The resulting residue was purified via silica gel chromatography (0-100% ethyl acetate/hexanes) to afford the title compound as a white solid contaminated with ˜12% of bisformylated material. The mixture was not purified further. LRMS (APCI) calc'd for C₅H₅BrN₂OS [M+1]⁺, [M+3]⁺: 220.9, 222.9, Found: 220.7, 222.7. ¹H NMR (500 MHz, d6-DMSO): δ 8.88 (s, 1H), 8.17 (s, 1H), 7.74 (s, 1H), 4.56 (d, 2H).

### Step 4: 3-Bromoimidazo[5,1-b][1,3]thiazole (17)

N-[(4-Bromo-1,3-thiazol-2-yl)methyl]formamide (3.7 g, 16.6 mmol) was taken up in phosphorous oxychloride (30 ml, 320 mmol). The mixture was heated at 100° C. for 3 hours. The reaction cooled to room temperature, poured over ice, and neutralized with 10 N and 6N aqueous sodium hydroxide. The resulting mixture was extracted with ethyl acetate (3×). The combined organics were dried over magnesium sulfate, filtered, and concentrated in vacuo to afford the title compound as a tan solid. LRMS (APCI) calc'd for C₅H₃BrN₂S [M+1]⁺, [M+3]⁺: 202.9, 204.9; Found: 202.7, 204.7. ¹H NMR (500 MHz, d6-DMSO): δ 8.17 (s, 1H), 7.42 (s, 1H), 7.17 (s, 1H).

### Step 5: 3-Bromo-7-nitroimidazo[5,1-b][1,3]thiazole (18)

In a dry flask, 3-bromoimidazo[5,1-b][1,3]thiazole (0.60 g, 3.0 mmol) was taken-up in acetonitrile (25 ml) under argon, and the mixture was cooled to −78° C. Nitronium tetrafluoroborate (0.5M in sulfolane) (15 ml, 7.5 mmol) was slowly added to the reaction mixture. The mixture was allowed to warm to room temperature over 60 minutes. The reaction was quenched with aqueous sodium bicarbonate and extracted three times with ethyl acetate. The combined organic extracts were washed with water (5×). The organic layer was dried over magnesium sulfate, filtered, dry-loaded onto 6 g of silica, and purified via silica gel chromatography (0-100% ethyl acetate/hexanes) to afford the title compound as a tan solid. LRMS (APCI) calc'd for C₅H₂BrN₃O₂S [M+1]⁺, [M+3]⁺: 247.9, 249.9; Found: 247.7, 249.6. ¹H NMR (500 MHz, d6-DMSO): δ 8.49 (s, 1H), 7.78 (s, 1H).

### Step 6: N-(3-Bromoimidazo[5,1-b][1,3]thiazol-7-yl)-2-(naphthalen-2-yl)acetamide (12)

3-Bromo-7-nitroimidazo[5,1-b][1,3]thiazole (120 mg, 0.50 mmol) and Platinum 3 wt % on activated carbon, doped with 0.6% Vanadium (160 mg, 0.03 mmol) were placed in a parr shaker vial that was covered with parafilm. Argon was introduced to the vial, and ethyl acetate (5 mL) was introduced via syringe. The vial was taken to the parr shaker and evacuated then backfilled with nitrogen (3×), then evacuated and backfilled with hydrogen (3×). The reaction was shaken under 50 psi hydrogen for 8 hours. It was then evacuated and backfilled with nitrogen (3×) and filtered through a celite plug, washing with ethyl acetate, to afford a cherry red solution. The solution was concentrated to ½ its original volume under reduced pressure (without using a water bath), and then immediately treated with naphthalen-2-ylacetyl chloride (120 mg, 0.60 mmol) and triethylamine (0.28 ml, 2.0 mmol). The resulting orange slurry stirred under a blanket of argon for 15 hours. The mixture was diluted with ethyl acetate and quenched with saturated aqueous ammonium chloride. The biphasic mixture was filtered and the solids collected to afford the title compound as a white powder. LRMS (APCI) calc'd for C₁₇H₁₂BrN₃OS [M+1]⁺, [M+3]⁺: 386.0, 388.0; Found: 385.7, 387.6. ¹H NMR (500 MHz, d6-DMSO): δ 10.95 (s, 1H), 7.98 (s, 1H), 7.86 (m, 3H), 7.80 (s, 1H), 7.47 (m, 3H), 7.27 (s, 1H), 3.77 (s, 2H).

### Example 84

### N-(3-cyanoimidazo[5,1-b][1,3]thiazol-7-yl)-2-(naphthalen-2-yl)acetamide (19)

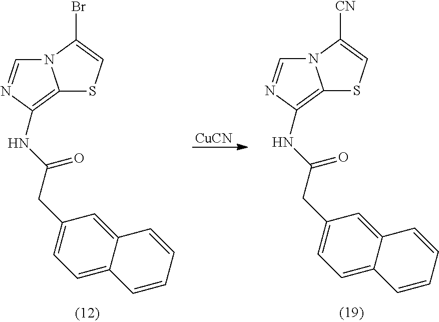 [SMILES: O=C(Cc1ccc2ccccc2c1)Nc1ncn2c(Br)csc12 ; "CuCN" ; N#Cc1csc2c(NC(=O)Cc3ccc4ccccc4c3)ncn12]

Copper(I) cyanide (16 mg, 0.18 mmol) and N-(3-bromoimidazo[5,1-b][1,3]thiazol-7-yl)-2-(naphthalen-2-yl)acetamide (27 mg, 0.07 mmol) were placed in a microwave vial that was placed under an argon atmosphere. N-Methyl-2-pyrrolidone (0.5 mL) was added. The vial was capped and the solution was heated in the microwave at 200° C. for 30 min. The reaction mixture was then diluted with ethyl acetate and dilute sodium bicarbonate. The aqueous layer was extracted with ethyl acetate (2×). The combined organics were washed with water (3×), dried over magnesium sulfate, filtered, and concentrated under reduced pressure. The resulting residue was purified via silica gel chromatography (75-100% ethyl acetate) to afford the title compound as a tan solid. LRMS (APCI) calc'd for C₁₈H₁₂N₄OS [M+H]⁺: 333.1, Found: 332.8. ¹H NMR (500 MHz, d6-DMSO): δ 11.07 (s, 1H), 8.36 (s, 1H), 8.20 (s, 1H), 7.86 (m, 3H), 7.80 (s, 1H), 7.47 (m, 3H), 3.79 (s, 2H).

### Example 85

### 7-[(Naphthalen-2-ylacetyl)amino]-3-(trifluoromethyl)imidazo[5,1-b][1,3]thiazole-2-carboxylic Acid (25)

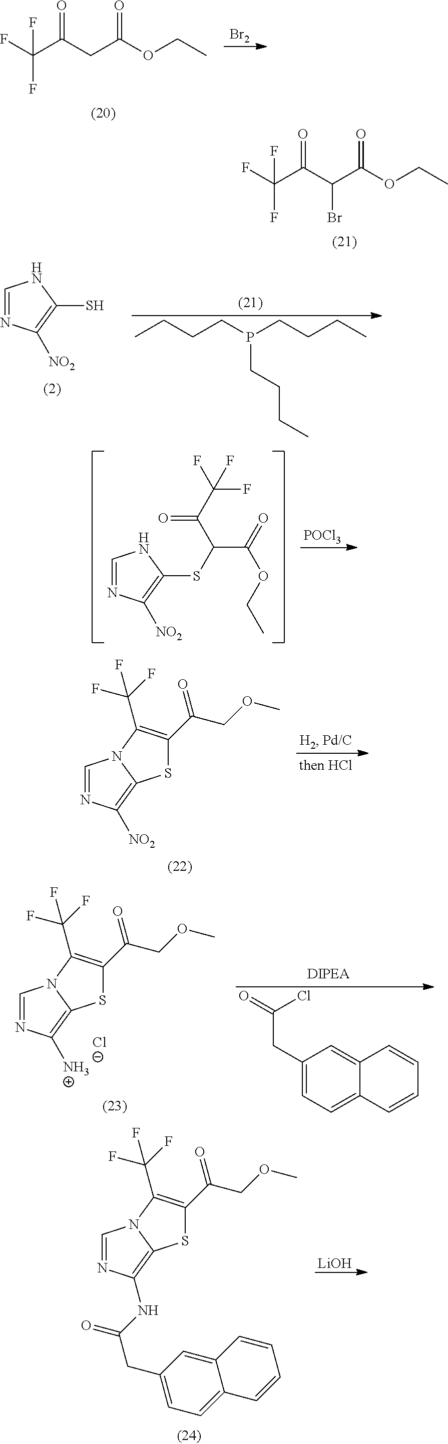

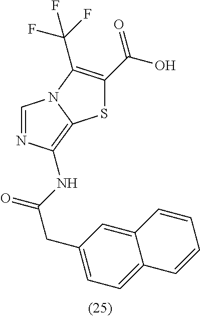 [SMILES: O=C(Cc1ccc2ccccc2c1)Nc1ncn2c(C(F)(F)F)c(C(=O)O)sc12]

### Step 1: Ethyl-2-Bromo-4,4,4-trifluoro-3-oxobutanoate (21)

Ethyl-4,4,4-trifluoro-3-oxobutanoate (10 g, 54.3 mmol) was added to CCl₄ (20 mL). Bromine (2.8 mL, 54.3 mmol) in CCl₄ (30 mL) was added dropwise via addition funnel over the period of one hour. The reaction was stirred for 18 hours at room temperature. The solvents were removed the residue was placed under vacuum to dry to afford the title compound.

### Step 2: 7-Nitro-3-(trifluoromethyl)imidazo[5,1-b][1,3]thiazole-2-carboxylate (22)

5-Nitro-1H-imidazole-4-thiol (5.0 g, 34 mmol) was placed in a vial that was evacuated and backfilled with nitrogen (3×). DMF (100 ml) and tributylphosphine (3.8 g, 17 mmol) were then added and the reaction stirred at room temperature for one hour. Ethyl-2-Bromo-4,4,4-trifluoro-3-oxobutanoate (9.06 g, 34.4 mmol) was then added and the reaction stirred at room temperature overnight. The mixture was then concentrated under reduced pressure and azeotroped with xylene two times to afford crude 4,4,4-Trifluoro-2-[(4-nitro-1H-imidazol-5-yl)sulfanyl]-3-oxobutanoate. To this residue was added phosphorous oxychloride (175 ml, 1900 mmol) and the reaction refluxed at 100° C. for 4 hrs. Excess phosphorous oxychloride was removed under reduced pressure. The resulting solution was slowly poured into ice to quench unreacted phosphorous oxychloride. Dichloromethane was then added to the oil and a precipitate formed. The precipitate was filtered and the solids collected were washed with dichloromethane and ethyl acetate. The combined organics were then concentrated down. The resulting residue was purified by silica gel chromatography eluting with dichloromethane in ethyl acetate to afford the title compound. LRMS (APCI) calc'd for C₉H₆F₃N₃O₄S [M+H]⁺: 310.2, Found: 310.0.

### Step 3: Ethyl 7-amino-3-(trifluoromethyl)imidazo[5,1-b][1,3]thiazole-2-carboxylate Hydrochloride (23)

7-Nitro-3-(trifluoromethyl)imidazo[5,1-b][1,3]thiazole-2-carboxylate (400 mg, 1.3 mmol) and 10 wt % palladium on carbon (140 mg, 0.13 mmol) were placed in a parr shaker vial. Ethyl acetate (7 ml) was added. The vial was taken to the parr shaker and evacuated then backfilled with nitrogen 3 times, then evacuated and backfilled with hydrogen 3 times. The reaction was shaken under 50-55 psi hydrogen for 7 hours. It was then filtered through a celite plug, washing with ethyl acetate (7 mL). 4 M HCl in dioxane (0.30 mL, 1.3 mmol) was then added to the filtrate and a precipitate immediately formed. The slurry was stirred for 5 minutes at room temperature. The solids were then collected by filtration, washed with DCM, and dried under vacuum overnight to afford the title compound. LRMS (APCI) calc'd for C₆H₄F₃N₃S [M+H]⁺: 208.0, Found: 207.8.

### Step 4: 7-[(Naphthalen-2-ylacetyl)amino]-3-(trifluoromethyl)imidazo[5,1-b][1,3]thiazole-2-carboxylate (24)

Ethyl 7-amino-3-(trifluoromethyl)imidazo[5,1-b][1,3]thiazole-2-carboxylate hydrochloride (80 mg, 0.25 mmol) and diisopropylethyl amine (140 uL, 1.0 mmol) were taken up in dichloromethane (1.0 mL). Naphthalen-2-ylacetyl chloride (62 mg, 0.30 mmol) in minimal dichloromethane was added dropwise and the reaction was allowed to stir for one hr. The reaction was then diluted with dichloromethane and transferred to a separatory funnel. The mixture was washed with saturated aqueous sodium bicarbonate, brine, dried with sodium sulfate, filtered, and concentrated under reduced pressure. The solid was then titurated with methanol and filtered to afford the title compound. LRMS (APCI) calc'd for C₂₁H₁₆F₃N₃O₃S [M+H]⁺: 448.4, Found: 447.9.

### Step 5: 7-[(Naphthalen-2-ylacetyl)amino]-3-(trifluoromethyl)imidazo[5,1-b][1,3]thiazole-2-carboxylic Acid (25)

7-[(Naphthalen-2-ylacetyl)amino]-3-(trifluoromethyl)imidazo[5,1-b][1,3]thiazole-2-carboxylate (63 mg, 0.14 mmol) was taken up in tetrahydrofuran (1.0 mL). Lithium hydroxide (1.0 M aqueous solution, 2.1 mL 0.21 mmol) was added dropwise and the suspension stirred for 16 hours at room temperature upon which time it became a colorless solution. The reaction was neutralized with acetic acid, and a precipitated formed. The solids were collected by filtration, washed with water and diethyl ether to afford the title compound. LRMS (APCI) calc'd for C₁₉H₁₂F₃N₃O₃S [M+H]⁺: 420.4, Found: 419.9. ¹H NMR (600 MHz, d6-DMSO): δ 11.10 (s, 1H), 8.17 (s, 1H), 7.84 (m, 3H), 7.78 (s, 1H), 7.46 (m, 3H), 3.78 (s, 2H).

### Example 86

### 7-[(Naphthalen-2-ylacetyl)amino]-3-(trifluoromethyl)imidazo[5,1-b][1,3]thiazole-2-carboxyamide (26)

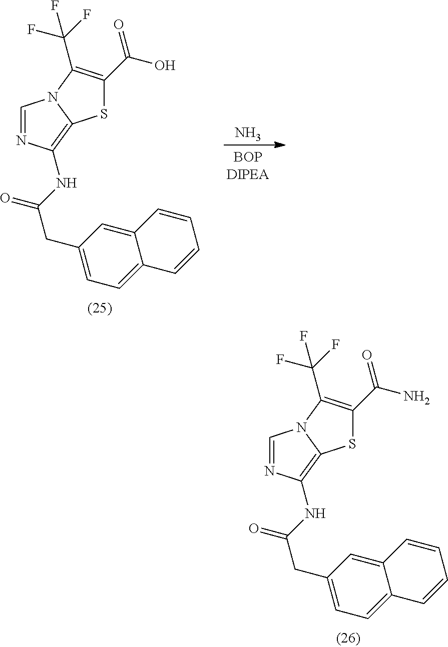

7-[(Naphthalen-2-ylacetyl)amino]-3-(trifluoromethyl)imidazo[5,1-b][1,3]thiazole-2-carboxylic acid (49 mg, 0.12 mmol), and diisopropylethyl amine (0.10 mL, 0.58 mmol) were taken up in N,N-dimethylformamide (3.0 mL) and allowed to stir at room temperature for five minutes. Then BOP (78 mg, 0.18 mmol) was added and the mixture was stirred an additional five minutes. 0.5 M ammonia in dioxane (2.3 mL, 1.17 mmol) was added and the reaction stirred at room temperature for 18 hours. Solvents were evaporated and the sample resuspended in DMF/MeOH for purification on RP-HPLC, water:acetonitrile+0.05% TFA modifier. Pure fractions were pooled and concentrated to afford the title compound as a neutral solid. LRMS (APCI) calc'd for C₁₉H₁₃F₃N₄O₂S [M+H]⁺: 419.4, Found: 418.9. ¹H NMR (600 MHz, d6-DMSO): δ 11.14, (s, 1H), 8.30 (s, 1H), 8.14 (s, 1H), 8.04 (s, 1H), 7.85 (m, 3H), 7.78 (s, 1H), 7.46 (m, 3H), 3.78 (s, 2H).

The utility of the compounds in accordance with the present invention as inhibitors of CDK5 may be demonstrated by methodology known in the art. Enzyme inhibition may be determined as follows.

### CDK5-p25 Kinase Enzymatic Assay

CDK5-p25 kinase (Invitrogen PV4677) enzymatic activity was measured using electrophoretic separation and fluorescent detection to monitor phosphorylation of a fluorescently labeled histone H1-derived peptide substrate, FL29 (5-FAM-GGGPATPKKAKKL-CONH2, Caliper #760429)

First, 0.25 uL of serially diluted compound in DMSO at 100× concentration was transferred into each well of a 384-well polystyrene assay plate. To this was added 15 uL of a 1.67× enzyme solution (500 pM enzyme, 1 mM DTT (Sigma D9779), 1× reconstitution buffer with protease inhibitor (Caliper #700329 proprietary formulation)), followed by centrifugation for 1 min at 1000 rpm, and incubation for 5-15 min at room temperature to allow binding of compound to enzyme. To initiate the reaction, 10 uL of substrate/ATP mix (245 mM Hepes pH 7.5, 0.003% Brij 35, 0.004% Tween, 12.75 uM ATP, 3.75 uM FL29 peptide) were added to each well, followed by centrifugation at 1000 rpm for 1 min. The reaction was then incubated at room temperature for 30 min. Final assay conditions were: 0.3 nM CDK5-p25, 1.5 uM peptide, 5 uM ATP, 0.6 mM DTT, 1% DMSO, 98 mM HEPES pH 7.5.

After incubation, reactions were quenched by the addition of 45 uL Caliper termination solution (Caliper proprietary formula), followed by centrifugation at 1000 rpm for 1 min. Extent of reaction (percentage conversion of substrate to phosphorylated product) was then measured using the Caliper EZ II reader. Dose-response curves were created by plotting the inhibition of enzyme activity (normalized relative to vehicle treated and no ATP control reactions) as a function of the log of compound concentration. The curve was analyzed using a four parameter logistical fit to calculate IC₅₀ values.

The IC50 potencies of the exemplary compounds of the invention were evaluated by the CDK5-p25 kinase enzymatic assay, as shown below:

| Example | <50 nM | 50 nM-99 nM | 100 nM-499 nM | 500 nM-3 μM |
|---|---|---|---|---|
| 1 | ✓ | | | |
| 2 | ✓ | | | |
| 3 | ✓ | | | |
| 4 | ✓ | | | |
| 5 | ✓ | | | |
| 6 | ✓ | | | |
| 7 | ✓ | | | |
| 8 | | | | ✓ |
| 9 | ✓ | | | |
| 10 | | | ✓ | |
| 11 | ✓ | | | |
| 12 | | ✓ | | |
| 13 | ✓ | | | |
| 14 | ✓ | | | |
| 15 | | | | ✓ |
| 16 | ✓ | | | |
| 17 | | ✓ | | |
| 18 | | | | ✓ |
| 19 | ✓ | | | |
| 20 | | ✓ | | |
| 21 | | | ✓ | |
| 22 | | | ✓ | |
| 23 | | ✓ | | |
| 24 | ✓ | | | |
| 25 | | | ✓ | |
| 26 | | ✓ | | |
| 27 | | ✓ | | |
| 28 | | | | ✓ |
| 29 | | | ✓ | |
| 30 | | ✓ | | |
| 31 | | ✓ | | |
| 32 | | | ✓ | |
| 33 | ✓ | | | |
| 34 | | | ✓ | |
| 35 | | ✓ | | |
| 36 | | ✓ | | |
| 37 | ✓ | | | |
| 38 | | | | ✓ |
| 39 | | ✓ | | |
| 40 | | | ✓ | |
| 41 | | ✓ | | |
| 42 | | ✓ | | |
| 43 | ✓ | | | |
| 44 | | ✓ | | |
| 45 | | ✓ | | |
| 46 | ✓ | | | |
| 47 | | | | ✓ |
| 48 | ✓ | | | |
| 49 | | ✓ | | |
| 50 | ✓ | | | |
| 51 | | | ✓ | |
| 52 | ✓ | | | |
| 53 | ✓ | | | |
| 54 | ✓ | | | |
| 55 | ✓ | | | |
| 56 | ✓ | | | |
| 57 | | ✓ | | |
| 58 | | | ✓ | |
| 59 | | ✓ | | |
| 60 | | ✓ | | |
| 61 | | ✓ | | |
| 62 | ✓ | | | |
| 63 | | ✓ | | |
| 64 | ✓ | | | |
| 65 | | | | ✓ |
| 66 | ✓ | | | |
| 67 | | | | ✓ |
| 68 | | | ✓ | |
| 69 | ✓ | | | |
| 70 | ✓ | | | |
| 71 | | ✓ | | |
| 72 | | | ✓ | |
| 73 | ✓ | | | |
| 74 | | ✓ | | |
| 75 | | | ✓ | |
| 76 | ✓ | | | |
| 77 | | ✓ | | |
| 78 | ✓ | | | |
| 79 | ✓ | | | |
| 80 | ✓ | | | |
| 81 | ✓ | | | |
| 82 | ✓ | | | ✓ |
| 83 | ✓ | | | |
| 84 | | | | ✓ |
| 85 | ✓ | | | |
| 86 | ✓ | | | |

### CDK5 Tau S235 Phosphorylation Assay in Rat Primary Cortical Neurons

Primary rat cortical neurons were plated at a density of 6000 cells/well in 384-well black/clear bottom Poly D-Lysine coated BD Falcon Biocoat plates using Neurobasal media supplemented with 1×B27+2 mM L-glutamine and 10% FBS. Cells were maintained at 37° C. and 5% CO₂ for 6 days in culture, with a one-half volume media change every 3-4 days. Prediluted compound in medium was added to cells in media at 1:5 dilution to create a 3-fold serial dilution series with 0.5% final DMSO concentration. Cells were incubated with compound for a total of 120 min at 37° C. After compound treatment, cells were washed with phosphate buffered saline (PBS), fixed 30 min at room temperature with 1% PFA diluted in PBS, and then washed three times again with PBS. Next, cells were permeabilized and blocked 1 hr at room temperature using 0.1% Triton X-100 and 5% normal goat serum. After permeabilization, the cells were washed 3× with PBS before incubation at 4° C. overnight with mouse anti-tau-3R (Upstate 05-803) (1:1000 final) and rabbit anti-tau-pS235 (ABR 38719) (1:500 final). The next day, cells were washed 4× with PBS and further incubated in the dark for 1 hr at room temperature with AlexaFluor goat anti mouse 488 (1:1000 final), AlexaFluor goat anti rabbit 594 (1:1000 final), and Hoechst 33342 dye (1:10000 final). Cells were washed 4× with PBS while protected from light and then imaged on an INCell Analyzer 1000 using a 10× objective. The phospho-S235 tau signal was calculated by measuring the average phospho-S235 signal for all cells expressing tau. The percentage inhibition of the pS235 signal relative to DMSO-treated controls was plotted as a function of the log of the compound dose and IC₅₀ values were calculated using a four parameter logistical fit of the data.

The compounds of the invention also demonstrated binding in off-target assays to the tau phosphorylation kinases GSKβ and CDK2.

Several methods for preparing the compounds of this invention are illustrated in the schemes and examples herein. Starting materials are made according to procedures known in the art or as illustrated herein. The following examples are provided so that the invention might be more fully understood. These examples are illustrative only and should not be construed as limiting the invention in any way.

The following abbreviations are used throughout the text:

Me: methyl

Et: ethyl

t-Bu: tert-butyl

Ar: aryl

Ph: phenyl

Bn: benzyl

Ac: acetyl

Dba: dibutylamine

EDC: 1-Ethyl-3-(3-dimethyllaminopropyl)carbodiimide

HOBt: 1-Hydroxybenzotriazole

rt: room temperature

HPLC: high performance liquid chromatography

X-Phos: 2-dicyclohexylphosphino-2′,4′,6′-triisopropylbiphenyl

TFA: trifluoroacetic acid

DMF: N,N-dimethylformamide

DMSO: dimethylsulfoxide

NEt₃: triethylamine

Pd₂(dba)₃: tris(dibenzylideneacetone)dipalladium(0)

BOP: Benzotriazole-1-yl-oxy-tris-(dimethylamino)-phosphonium hexafluorophosphate

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

1. A compound of formula (I): 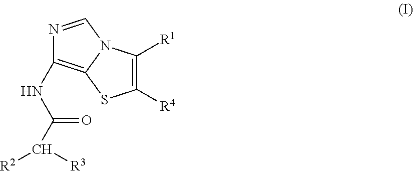
wherein:
R¹ is selected from the group consisting of
(1) hydrogen,
(2) halogen,
(3) cyano,
(4) —C₁₋₃ alkyl, optionally substituted with one or more fluoro, or
(5) —C₃₋₈ cycloalkyl;
R² is selected from the group consisting of
(1) hydrogen,
(2) halogen,
(2) —C₁₋₆ alkyl, optionally substituted with hydroxyl, or
(4) hydroxyl;
R³ is selected from the group consisting of
(1) —C₃₋₈ cycloalkyl,
(2) —C₆₋₁₀ aryl,
(3) heteroaryl, which is a cyclic or polycyclic group having 5 to 12 ring atoms, said ring atoms selected from C, C(═O), and at least one heteroatom selected from N, O or S, wherein at least one of the rings is aromatic,
(4) a heterocyclic group having 4 to 8 ring atoms, wherein one ring atom is a heteroatom selected from the group consisting of nitrogen, sulfur or oxygen,
wherein said R⁵ cycloalkyl, heterocyclic, aryl or heteroaryl moiety is optionally substituted with one or more
(a) halogen,
(b) cyano,
(c) —O—C₁₋₆ alkyl,
(d) —C₁₋₆ alkyl,
(e) OH,
(f) —NR⁶R⁷,
(g) heteroaryl, which is a cyclic or polycyclic group having 5 to 12 ring atoms, said ring atoms selected from C, C(═O), and at least one heteroatom selected from N, O or S, wherein at least one of the rings is aromatic,
(h) —C₆₋₁₀ aryl,
(i) —NH—C(═O)—R⁵, or
(j) —S(═O)₂—R⁵,
wherein said alkyl, aryl or heteroaryl is optionally substituted with one or more
(i) —C₁₋₆ alkyl,
(ii) —OC₁₋₆ alkyl,
(iii) NR⁸R⁹;
R⁴ is selected from the group consisting of
(1) hydrogen,
(2) —C(═O)—OH, or
(3) —C(═O)—NH₂;
R⁵ is selected from the group consisting of
(1) hydrogen,
(2) —C₁₋₆ alkyl,
(3) —C₆₋₁₀ aryl, or
(4) heteroaryl, which is a cyclic or polycyclic group having 5 to 12 ring atoms, said ring atoms selected from C, C(═O), and at least one heteroatom selected from N, O or S, wherein at least one of the rings is aromatic;
R⁶, R⁷, R⁸ and R⁹ are selected from the group consisting of
(1) hydrogen,
(2) —C₁₋₆ alkyl,
(3) —C₆₋₁₀ aryl, or
(4) heteroaryl, which is a cyclic or polycyclic group having 5 to 12 ring atoms, said ring atoms selected from C, C(═O), and at least one heteroatom selected from N, O or S, wherein at least one of the rings is aromatic,
wherein said alkyl, aryl or heteroaryl moiety is optionally substituted with one or more
(a) halogen
(b) cyano
(c) —O—C₁₋₆ alkyl,
(d) —C₁₋₆ alkyl,
(e) OH,
(f) —NR¹⁰R¹¹,
or R⁶ and R⁷, or R⁸ and R⁹, may be linked together with the nitrogen to which they are both attached to form a non-aromatic cyclic ring having from 5 to 12 ring atoms selected from C, N, O, S, S═O or SO₂, wherein said cyclic ring is optionally substituted with one or more
(a) halogen
(b) cyano
(c) —C₃₋₈ cycloalkyl
(d) —O—C₁₋₆ alkyl, or
(e) —C₁₋₆ alkyl;
R¹⁰ and R¹¹ are selected from the group consisting of
(1) hydrogen, and
(2) —C₁₋₆ alkyl.
or a pharmaceutically acceptable salt thereof.

2. A compound of claim 1, wherein R¹ is —C₁₋₃ alkyl, optionally substituted with one or more fluoro.

3. A compound of claim 2, wherein R¹ is —CF₃.

4. A compound of claim 3, wherein R² is hydrogen.

5. A compound of claim 4, wherein R³ is aryl, which is optionally substituted by
(a) halogen,
(b) —O—C₁₋₆ alkyl,
(c) —C₁₋₆ alkyl,
(d) —NR⁶R⁷,
(e) heteroaryl group selected from pyrazolyl, pyridinyl, imidazolyl, pyridazinyl, pyrazinyl, pyrimidinyl, benzodioxol, indazolyl, quinolinyl, thienyl,
(f) —C₆₋₁₀ aryl selected from phenyl or napthyl,
(g) —NH—C(═O)—R⁵, wherein R⁵ is aryl or heteroaryl, or
(h) —SO₂—C₁₋₆ alkyl,
wherein said alkyl, aryl or heteroaryl is optionally substituted with one or more
(i) —C₁₋₆ alkyl,
(ii) —OC₁₋₆ alkyl,
(iii) NR⁸R⁹.

6. A compound of claim 4, wherein R³ is heteroaryl, selected from the group consisting of quinolinyl, pyridinyl, imidazolyl, imidazo[1,2-a]pyrimidinyl, imidazo[1,2-a]pyridinyl, benzodioxolyl, pyrimidinyl, indazolyl, indolyl and isoindolyl, wherein said heteroaryl group is optionally substituted by one or more
(a) halogen
(b) —C₁₋₆ alkyl,
(c) heteroaryl, which is a cyclic or polycyclic group having 5 to 12 ring atoms, said ring atoms selected from C, C(═O), and at least one heteroatom selected from N, O or S, wherein at least one of the rings is aromatic,
(d) —C₆₋₁₀ aryl selected from phenyl or napthyl,
wherein said alkyl, aryl or heteroaryl is optionally substituted with one or more
(i) —C₁₋₆ alkyl,
(ii) —OC₁₋₆ alkyl, or
(iii) NR⁸R⁹.

7. A compound of claim 6, wherein R⁴ is —C(═O)—OH or —C(═O)—NH₂.

8. A compound of claim 1, wherein the compound of formula (I) is a compound of formula (II): 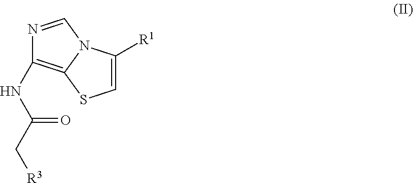
or a pharmaceutically acceptable salt thereof.

9. A compound of claim 8, wherein R¹ is —C₁₋₃ alkyl, optionally substituted with one or more fluoro.

10. A compound of claim 9, wherein R¹ is —CF₃.

11. A compound of claim 10, wherein R³ is aryl, which is optionally substituted by
(a) halogen,
(b) —O—C₁₋₆ alkyl,
(c) —C₁₋₆ alkyl,
(d) —NR⁶R⁷,
(e) heteroaryl, which is selected from pyrazolyl, pyridinyl, imidazolyl, pyridazinyl, pyrazinyl, pyrimidinyl, benzodioxol, indazolyl, quinolinyl, thienyl,
(f) —C₆₋₁₀ aryl, which is selected from phenyl or napthyl,
(g) —NH—C(═O)—R⁵, wherein R⁵ is aryl or heteroaryl, or
(h) —SO₂—C₁₋₆ alkyl,
wherein said alkyl, aryl or heteroaryl is optionally substituted with one or more
(i) —C₁₋₆ alkyl,
(ii) —OC₁₋₆ alkyl,
(iii) NR⁸R⁹.

12. A compound of claim 10, wherein R³ is heteroaryl, which is selected from quinolinyl, pyridinyl, imidazolyl, imidazo[1,2-a]pyrimidinyl, imidazo[1,2-a]pyridinyl, benzodioxolyl, pyrimidinyl, indazolyl, indolyl and isoindolyl, and the heteroaryl group is optionally substituted with one or more
(a) halogen
(b) —C₁₋₆ alkyl,
(c) heteroaryl, which is a cyclic or polycyclic group having 5 to 12 ring atoms, said ring atoms selected from C, C(═O), and at least one heteroatom selected from N, O or S, wherein at least one of the rings is aromatic, or
(d) —C₆₋₁₀ aryl,
wherein said alkyl, aryl or heteroaryl is optionally substituted with one or more
(i) —C₁₋₆ alkyl,
(ii) —OC₁₋₆ alkyl,
(iii) NR⁸R⁹.

13. A compound of claim 1, wherein the compound of formula (I) is a compound of formula (III): 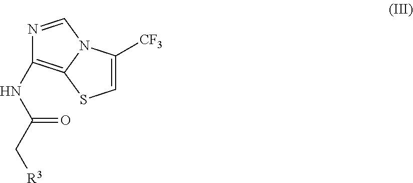
or a pharmaceutically acceptable salt thereof.

14. A compound of claim 13, wherein R³ is aryl selected from phenyl and napthyl, wherein the aryl is optionally substituted by one or more
(a) halogen,
(b) —O—C₁₋₆ alkyl,
(c) —C₁₋₆ alkyl,
(d) —NR⁶R⁷;
(e) heteroaryl, selected from pyrazolyl, pyridinyl, imidazolyl, pyridazinyl, pyrazinyl, pyrimidinyl, benzodioxol, indazolyl, quinolinyl and thienyl,
(f) —C₆₋₁₀ aryl, selected from phenyl or napthyl,
(g) —NH—C(═O)—R⁵, wherein R⁵ is aryl or heteroaryl, or
(h) —SO₂—C₁₋₆ alkyl,
wherein said alkyl, aryl or heteroaryl is optionally substituted with one or more
(i) —C₁₋₆ alkyl,
(ii) —OC₁₋₆ alkyl,
(iii) NR⁸R⁹.

15. A compound of claim 13, wherein R³ is heteroaryl selected from quinolinyl, pyridinyl, imidazolyl, imidazo[1,2-a]pyrimidinyl, imidazo[1,2-a]pyridinyl, benzodioxolyl, pyrimidinyl, indazolyl, indolyl and isoindolyl, wherein the heteroaryl group is optionally substituted by one or more
(a) halogen
(b) —C₁₋₆ alkyl,
(c) heteroaryl, which is a cyclic or polycyclic group having 5 to 12 ring atoms, said ring atoms selected from C, C(═O), and at least one heteroatom selected from N, O or S, wherein at least one of the rings is aromatic,
(d) —C₆₋₁₀ aryl,
wherein said alkyl, aryl or heteroaryl is optionally substituted with one or more
(i) —C₁₋₆ alkyl,
(ii) —OC₁₋₆ alkyl,
(iii) NR⁸R⁹.

16. A compound of claim 1, which is selected from the group consisting of
2-[4-(2-Oxo-1,3-oxazolidin-3-yl)phenyl]-N-[3-(trifluoromethyl)imidazo[5,1-b][1,3]thiazol-7-yl]acetamide;
2-(2-naphthyl)-N-[3-(trifluoromethyl)imidazo[5,1-b][1,3]thiazol-7-yl]acetamide;
2-quinolin-6-yl-N-[3-(trifluoromethyl)imidazo[5,1-b][1,3]thiazol-7-yl]acetamide;
2-isoquinolin-7-yl-N-[3-(trifluoromethyl)imidazo[5,1-b][1,3]thiazol-7-yl]acetamide;
2-isoquinolin-6-yl-N-[3-(trifluoromethyl)imidazo[5,1-b][1,3]thiazol-7-yl]acetamide;
2-(6-methoxy-2-naphthyl)-N-[3-(trifluoromethyl)imidazo[5,1-b][1,3]thiazol-7-yl]acetamide;
2-(6-chloropyridin-3-yl)-N-[3-(trifluoromethyl)imidazo[5,1-b][1,3]thiazol-7-yl]acetamide;
2-isoquinolin-3-yl-N-[3-(trifluoromethyl)imidazo[5,1-b][1,3]thiazol-7-yl]acetamide;
2-(7-methoxy-2-naphthyl)-N-[3-(trifluoromethyl)imidazo[5,1-b][1,3]thiazol-7-yl]acetamide;
2-(3′-methoxybiphenyl-4-yl)-N-[3-(trifluoromethyl)imidazo[5,1-b][1,3]thiazol-7-yl]acetamide;
2-(4-pyridin-3-ylphenyl)-N-[3-(trifluoromethyl)imidazo[5,1-b][1,3]thiazol-7-yl]acetamide;
2-(4-pyridin-4-ylphenyl)-N-[3-(trifluoromethyl)imidazo[5,1-b][1,3]thiazol-7-yl]acetamide;
2-quinolin-7-yl-N-[3-(trifluoromethyl)imidazo[5,1-b][1,3]thiazol-7-yl]acetamide;
2-(5-methoxy-2-naphthyl)-N-[3-(trifluoromethyl)imidazo[5,1-b][1,3]thiazol-7-yl]acetamide;
2-amino-2-(2-naphthyl)-N-[3-(trifluoromethyl)imidazo[5,1-b][1,3]thiazol-7-yl]acetamide;
2-[4-(2-oxo-1,3-oxazolidin-3-yl)phenyl]-N-[3-(trifluoromethyl)imidazo[5,1-b][1,3]thiazol-7-yl]acetamide;
2-[4-(1H-pyrazol-4-yl)phenyl]-N-[3-(trifluoromethyl)imidazo[5,1-b][1,3]thiazol-7-yl]acetamide;
2-[4-(2-oxopyrrolidin-1-yl)phenyl]-N-[3-(trifluoromethyl)imidazo[5,1-b][1,3]thiazol-7-yl]acetamide;
2-(6-methoxy-2-naphthyl)-N-[3-(trifluoromethyl)imidazo[5,1-b][1,3]thiazol-7-yl]propanamide;
2-[4-(1H-pyrazol-3-yl)phenyl]-N-[3-(trifluoromethyl)imidazo[5,1-b][1,3]thiazol-7-yl]acetamide;
2-[4-(1-methyl-1H-pyrazol-4-yl)phenyl]-N-[3-(trifluoromethyl)imidazo[5,1-b][1,3]thiazol-7-yl]acetamide;
2-(tetrahydro-2H-pyran-4-yl)-N-[3-(trifluoromethyl)imidazo[5,1-b][1,3]thiazol-7-yl]acetamide;
2-(3-methylisoxazol-5-yl)-N-[3-(trifluoromethyl)imidazo[5,1-b][1,3]thiazol-7-yl]acetamide;
2-[4-(1H-imidazol-1-yl)phenyl]-N-[3-(trifluoromethyl)imidazo[5,1-b][1,3]thiazol-7-yl]acetamide;
2-[3-(1H-pyrazol-4-yl)phenyl]-N-[3-(trifluoromethyl)imidazo[5,1-b][1,3]thiazol-7-yl]acetamide;
2-(trans-4-aminocyclohexyl)-N-[3-(trifluoromethyl)imidazo[5,1-b][1,3]thiazol-7-yl]acetamide;
7-[(2-naphthylacetyl)amino]-3-(trifluoromethyl)imidazo[5,1-b][1,3]thiazole-2-carboxylic acid;
2-(4-pyridazin-4-ylphenyl)-N-[3-(trifluoromethyl)imidazo[5,1-b][1,3]thiazol-7-yl]acetamide;
7-[(2-naphthylacetyl)amino]-3-(trifluoromethyl)imidazo[5,1-b][1,3]thiazole-2-carboxamide;
2-[4-(aminomethyl)phenyl]-N-[3-(trifluoromethyl)imidazo[5,1-b][1,3]thiazol-7-yl]acetamide;
N-imidazo[5,1-b][1,3]thiazol-7-yl-2-(2-naphthyl)acetamide;
2-imidazo[1,2-a]pyrimidin-2-yl-N-[3-(trifluoromethyl)imidazo[5,1-b][1,3]thiazol-7-yl]acetamide;
2-imidazo[1,2-a]pyridin-2-yl-N-[3-(trifluoromethyl)imidazo[5,1-b][1,3]thiazol-7-yl]acetamide;
2-(1,3-benzodioxol-5-yl)-N-[3-(trifluoromethyl)imidazo[5,1-b][1,3]thiazol-7-yl]acetamide;
2-[4-(dimethylamino)phenyl]-N-[3-(trifluoromethyl)imidazo[5,1-b][1,3]thiazol-7-yl]acetamide;
2-pyrimidin-5-yl-N-[3-(trifluoromethyl)imidazo[5,1-b][1,3]thiazol-7-yl]acetamide;
2-fluoro-2-(2-naphthyl)-N-[3-(trifluoromethyl)imidazo[5,1-b][1,3]thiazol-7-yl]acetamide;
2-(2-naphthyl)-N-[3-(trifluoromethyl)imidazo[5,1-b][1,3]thiazol-7-yl]propanamide;
2-[4-(2-aminopyrimidin-5-yl)phenyl]-N-[3-(trifluoromethyl)imidazo[5,1-b][1,3]thiazol-7-yl]acetamide;
(2S)-2-(2-naphthyl)-N-[3-(trifluoromethyl)imidazo[5,1-b][1,3]thiazol-7-yl]propanamide;
2-(1H-indazol-5-yl)-N-[3-(trifluoromethyl)imidazo[5,1-b][1,3]thiazol-7-yl]acetamide;
N-(3-cyclopropylimidazo[5,1-b][1,3]thiazol-7-yl)-2-(2-naphthyl)acetamide;
2-quinolin-6-yl-N-[3-(trifluoromethyl)imidazo[5,1-b][1,3]thiazol-7-yl]propanamide;
2-hydroxy-2-(2-naphthyl)-N-[3-(trifluoromethyl)imidazo[5,1-b][1,3]thiazol-7-yl]acetamide;
N-[3-(difluoromethyl)imidazo[5,1-b][1,3]thiazol-7-yl]-2-(2-naphthyl)acetamide;
2-chloro-2-(2-naphthyl)-N-[3-(trifluoromethyl)imidazo[5,1-b][1,3]thiazol-7-yl]acetamide;
2-(4-chlorophenyl)-N-[3-(trifluoromethyl)imidazo[5,1-b][1,3]thiazol-7-yl]acetamide;
(R or S)-2-hydroxy-2-(2-naphthyl)-N-[3-(trifluoromethyl)imidazo[5,1-b][1,3]thiazol-7-yl]acetamide;
(R or S)-2-chloro-2-(2-naphthyl)-N-[3-(trifluoromethyl)imidazo[5,1-b][1,3]thiazol-7-yl]acetamide;
(2S)-2-quinolin-6-yl-N-[3-(trifluoromethyl)imidazo[5,1-b][1,3]thiazol-7-yl]propanamide;
(2R)-2-quinolin-6-yl-N-[3-(trifluoromethyl)imidazo[5,1-b][1,3]thiazol-7-yl]propanamide;
2-[3-(1H-pyrazol-3-yl)phenyl]-N-[3-(trifluoromethyl)imidazo[5,1-b][1,3]thiazol-7-yl]acetamide;
2-{3-[(2-aminopyridin-4-yl)amino]phenyl}-N-[3-(trifluoromethyl)imidazo[5,1-b][1,3]thiazol-7-yl]acetamide;
2-(6-phenylpyridin-3-yl)-N-[3-(trifluoromethyl)imidazo[5,1-b][1,3]thiazol-7-yl]acetamide;
2-[4-(5-methylpyrazin-2-yl)phenyl]-N-[3-(trifluoromethyl)imidazo[5,1-b][1,3]thiazol-7-yl]acetamide;
2-(3-pyridin-3-ylphenyl)-N-[3-(trifluoromethyl)imidazo[5,1-b][1,3]thiazol-7-yl]acetamide;
2-[4-(3-thienyl)phenyl]-N-[3-(trifluoromethyl)imidazo[5,1-b][1,3]thiazol-7-yl]acetamide;
2-(4-pyridin-2-ylphenyl)-N-[3-(trifluoromethyl)imidazo[5,1-b][1,3]thiazol-7-yl]acetamide;
2-[4-(6-methylpyridin-3-yl)phenyl]-N-[3-(trifluoromethyl)imidazo[5,1-b][1,3]thiazol-7-yl]acetamide;
2-(3-pyridin-2-ylphenyl)-N-[3-(trifluoromethyl)imidazo[5,1-b][1,3]thiazol-7-yl]acetamide;
2-(1H-indol-3-yl)-N-[3-(trifluoromethyl)imidazo[5,1-b][1,3]thiazol-7-yl]acetamide;
2-[4-(methylsulfonyl)phenyl]-N-[3-(trifluoromethyl)imidazo[5,1-b][1,3]thiazol-7-yl]acetamide;
N-[3-(2-oxo-2-{[3-(trifluoromethyl)imidazo[5,1-b][1,3]thiazol-7-yl]amino}ethyl)phenyl]isonicotinamide;
N-[3-(2-oxo-2-{[3-(trifluoromethyl)imidazo[5,1-b][1,3]thiazol-7-yl]amino}ethyl)phenyl]isonicotinamide;
N-[4-(2-oxo-2-{[3-(trifluoromethyl)imidazo[5,1-b][1,3]thiazol-7-yl]amino}ethyl)phenyl]nicotinamide;
N-[4-(2-oxo-2-{[3-(trifluoromethyl)imidazo[5,1-b][1,3]thiazol-7-yl]amino}ethyl)phenyl]isonicotinamide;
2-[6-(2-aminophenyl)pyridin-3-yl]-N-[3-(trifluoromethyl)imidazo[5,1-b][1,3]thiazol-7-yl]acetamide;
2-(3-pyridin-4-ylphenyl)-N-[3-(trifluoromethyl)imidazo[5,1-b][1,3]thiazol-7-yl]acetamide;
N-(3-methylimidazo[5,1-b][1,3]thiazol-7-yl)-2-(2-naphthyl)acetamide;
2-(1-naphthyl)-N-[3-(trifluoromethyl)imidazo[5,1-b][1,3]thiazol-7-yl]acetamide;
3-hydroxy-2-(2-naphthyl)-N-[3-(trifluoromethyl)imidazo[5,1-b][1,3]thiazol-7-yl]propanamide;
2-(1,3-dihydro-2H-isoindol-2-yl)-N-[3-(trifluoromethyl)imidazo[5,1-b][1,3]thiazol-7-yl]acetamide;
N-(3-bromoimidazo[5,1-b][1,3]thiazol-7-yl)-2-(2-naphthyl)acetamide;
N-(3-cyanoimidazo[5,1-b][1,3]thiazol-7-yl)-2-(2-naphthyl)acetamide;
or a pharmaceutically acceptable salt thereof.

17. A pharmaceutical composition which comprises a compound of claim 1, or a pharmaceutically acceptable salt thereof, and a pharmaceutically acceptable carrier.

18. A method of treating Alzheimer's Disease in a patient, comprising the step of administering to the patient an effective amount of a compound of claim 1, or a pharmaceutically acceptable salt thereof.

19. (canceled)

20. (canceled)

21. A method of treating a disease or disorder in which tau phosphorylation kinases are implicated, comprising the step of administering to the patient an effective amount of a compound of claim 1, or a pharmaceutically acceptable salt thereof.

22. (canceled)

23. (canceled)

24. A method for the treatment of cancer comprising the step of administering to the patient an effective amount of a compound of claim 1.

25. A method of treating a disease or disorder in which cell cycle regulation kinases are implicated, comprising the step of administering to the patient an effective amount of a compound of claim 1, or a pharmaceutically acceptable salt thereof.

26. (canceled)

27. (canceled)

28. A compound of claim 5, wherein R⁴ is —C(═O)—OH or —C(═O)—NH₂.
